(19) United States
(12) Patent Application Publication
(10) Pub. No.: US 2024/0156083 A1
(43) Pub. Date: May 16, 2024

(54) CRYOBIOPRINTING FOR TISSUE FABRICATION AND STORAGE

(71) Applicant: THE BRIGHAM AND WOMEN'S HOSPITAL, INC., Boston, MA (US)

(72) Inventors: Yu Shrike Zhang, Boston, MA (US); Zeyu Luo, Boston, MA (US)

(21) Appl. No.: 18/506,291

(22) Filed: Nov. 10, 2023

Related U.S. Application Data
(60) Provisional application No. 63/424,198, filed on Nov. 10, 2022.

(51) Int. Cl. A01N 1/02; C12N 5/077; C12N 5/0775; C12N 5/079

(52) U.S. Cl. CPC A01N 1/0284; A01N 1/0221; C12N 5/0618; C12N 5/0656; C12N 5/0658; C12N 5/0662; C12N 2513/00; C12N 2533/54; C12N 2537/10

(57) ABSTRACT

A method of making a 3D-printed biomaterial is described. The method includes depositing a hydrogel comprising cells and a cryoprotectant from a 3D printer onto a freezing plate to form a frozen hydrogel filament; depositing additional hydrogel comprising cells and a cryoprotectant from a 3D printer in contact with a previously placed frozen hydrogel filament to form a 3D-printed biomaterial comprising a plurality of frozen hydrogel filaments; and removing the 3D-printed biomaterial from the freezing plate. 3D-printed biomaterials made by the method are also described.

### CONTINUING APPLICATION DATA

This application claims the benefit of U.S. Provisional Application Ser. No. 63/424,198, filed Nov. 10, 2022, the disclosure of which are incorporated by reference herein.

### STATEMENT REGARDING FEDERALLY SPONSORED RESEARCH

This invention was made with government support under Grant Number R56EB034702, awarded by the National Institutes of Health. The Government has certain rights in this invention.

### BACKGROUND

Three-dimensional (3D) bioprinting has paved a new avenue for fabricating intricate cell-laden tissue constructs. So far, bioprinting has been adopted in myriad applications such as tissue engineering, tissue model engineering, and organoid production, among others. There are, however, challenges regarding the fabrication and storage of shelf-ready 3D-bioprinted tissue constructs. Due to the intrinsic complexities involved in most bioprinting processes, including the broadly adopted extrusion bioprinting, using this method as an on-site fabrication technique can be cumbersome or sometimes impractical under a number of scenarios. Furthermore, the lack of a functional approach for long-term storage of cell-laden tissue constructs (Jeon O., et al., Mater. Today Chem 12, 61-70) precludes the shelf-availability of pre-made bioprinted products.

Combining the fabrication and storage methods (i.e., bioprinting and cryopreservation) is a potential solution to address the obstacles mentioned above. As such, the bioprinted tissues can be fabricated in advance off-site, allowing direct storage in cryogenic conditions for later academic or clinical applications. The main challenge that needs to be addressed is ice crystal-formation and recrystallization, which may negatively affect cell viability in the fabricated products. The bioink components should be properly designed to efficiently preserve cells during bioprinting and storage/resuscitation periods. Various polymers, such as poly(vinyl alcohol) and poly(ethylene glycol), either in the form of bulk (Mitchell DE, et al., Mater. Horizons 6, 364-368) or self-assembled nanoparticles (Georgiou PG, et al., J. Am. Chem. Soc 143, 7449-7461), have shown promising capability in inhibiting ice-recrystallization.

Cryoprotective agents (CPAs) are also believed to have a critical effect on eliminating or minimizing ice crystal-formation (Jiang S, et al., Nat. Commun 10, 1-14). In the absence of CPAs, the ice crystals grow within the scaffold and may jeopardize cell viability by damaging the cell membranes over the freezing/thawing cycles (Huang H, et al., ACS Biomater. Sci. Eng 3, 1758-1768). Dimethylsulfoxide (DMSO) is a traditional CPA widely used in cryopreservation to minimize ice crystal-formation (Bahari L, et al., PLoS One. 13, e0192265). Besides, CPAs with larger molecular weights can protect the cells from osmotic damages caused by extracellular hypertonic and hypotonic conditions over the freezing and thawing periods (Solocinski J, et al., Cryobiology. 75, 134-143). Among these CPAs, various disaccharides such as sucrose (Tsai S, et al., Rev Aquac. 10, 703-715), trehalose (Malo C, et al., Cryobiology. 61, 17-21), lactose (Silva CG, et al., Cryobiology. 70, 90-94), and maltose (Hino T, et al., Reprod. Med. Biol 6, 229-233) together with trisaccharides such as melezitose (Hino T, et al., Reprod. Med. Biol 6, 229-233) and raffinose (Eroglu A, Cryobiology. 60, S54—S59) have shown promising cryoprotective effects.

There remains a need for improved methods of fabricating cell-laden tissue constructs using the combination of bioprinting and cryopreservation.

### SUMMARY

One significant drawback of existing bioprinted tissues is their lack of shelf-availability caused by complications in both fabrication and storage. A cryobioprinting strategy for simultaneously fabricating and storing cell-laden volumetric tissue constructs through seamlessly combining extrusion bioprinting and cryopreservation is described herein. The cryobioprinting performance was investigated by designing, fabricating, and storing cell-laden constructs made of our optimized cryoprotective gelatin-based bioinks using a freezing plate with precisely controllable temperature. The in situ freezing process further promoted the printability of cell-laden hydrogel bioinks to achieve freeform structures otherwise inconvenient with direct extrusion bioprinting. The effects of bioink composition on printability and cell viability were evaluated. The functionality of the method was finally investigated using cell differentiation and chick ex ovo assays. The results confirmed the feasibility and efficacy of cryobioprinting as a single-step method for concurrent tissue biofabrication and storage.

In addition, due to the poor mechanical properties of many hydrogel bioinks, conventional three-dimensional (3D) extrusion bioprinting is usually conducted based on the X-Y plane, where the deposited layers are stacked in the Z direction with or without the support of prior layers. Herein, we report a technique taking advantage of a cryoprotected bioink to enable direct extrusion bioprinting in the vertical direction in the presence of cells, using a freezing plate with precise temperature-control. Of interest, vertical 3D cryobioprinting concurrently allows the user to create freestanding filamentous constructs containing interconnected, anisotropic microchannels featuring gradient sizes aligned in the vertical direction, also associated with enhanced mechanical performances. Skeletal myoblasts within the 3D-cryobioprinted hydrogel constructs show enhanced alignment compared to the same cells in the standard hydrogel constructs. This method is further extended to a multi-material format, finding potential applications in interface tissue engineering, such as creation of the muscle-tendon unit and the muscle-microvascular unit. The unique vertical 3D cryobioprinting technique presented here suggests improvements in robustness and versatility to engineer certain tissue types especially those anisotropic in nature, and may extend broad utilities in tissue engineering, regenerative medicine, drug discovery, and personalized therapeutics.

### BRIEF DESCRIPTION OF THE FIGURES

FIG. 1 provides schematic illustration of cryobioprinting of tissue constructs for simultaneous cryopreservation. The cell-laden bioink is cryobioprinted on a freezing plate with tightly controlled temperature and kept in cryogenic conditions for long(er)-term preservation. The cryobioprinted structure can be then transferred, revived, and crosslinked immediately before subsequent culturing to achieve tissue-formation at the desired usage site. Figure created using BioRender.

FIGS. 2A-2V provide sample cryobioprinted constructs and simulation results for fabricating a two-layer grid structure. (A to R), Cryobioprinted 2D patterns, 3D structures, and freeform multi-material models made of (A to F) GelMA, (G to L) gelatin, and (M to R) GelMA+gelatin. Scale bars: 5 mm (S), Temperature gradient in the first layer of a grid structure during cryobioprinting. The initial temperature of the bioink was set to 15° C., and the freezing plate was simulated as a constant −15° C. surface. Panels (a) to (c) demonstrate the progress of the cryobioprinting, and panel (d) exhibits the freezing frontier lines in the experiment and the simulation. (T), Temperature gradient in the second layer of the grid structure. The regions that were in contact with the first layer froze faster. (U), Temperature changes through the thickness of the first layer during cryobioprinting. At 0.09 s after the bioink touched the freezing plate, the entire cross-section was frozen, and after 0.45 s, the bioink surface temperature reached approximately −14.5° C. Since the dominant mechanism of heat-transfer through the thickness was conduction, the temperature change was linear. (V), Comparing the average temperature over the freezing period for the first and the second layers using different values of Tₚ.

FIGS. 3A-3J provide cell viability in cryopreserved GelMA constructs with different concentrations and types of CPAs using seven different cell types. (A), Representative fluorescence images of the C2C12 cells encapsulated in bioinks, cryopreserved for 72 h, and resuscitated before staining/imaging. The cryopreserved bioinks were prepared using 5% (w/v) GelMA, different concentrations of DMSO, and different concentrations of saccharides. (B to D), Quantified increase in cell viability post-cryopreservation for 72 h due to supplementing the cryoprotective bioink with various saccharides: (B), Raffinose (Raf.). (C), Lactose (Lac.). (D), Melezitose (Mel.). (E to J), Significant increase in cell viability of various cell types when selected CPAs were employed. n=3. (Ctrl: Control)

FIGS. 4A-4I provide (A), Representative live (green)/dead (red) fluorescence images of hMSCs encapsulated in the three selected cryoprotective bioinks and cryopreserved for 72 h. (B to D), Osteogenic differentiation: (B), fluorescence micrographs, (C), semi-quantitative measurements of RUNX2 (1 week) and osteocalcin (2 and 3 weeks), and (D), quantification of Alizarin Red S-staining (3 weeks). (E to G), Chondrogenic differentiation: (E), fluorescence micrographs, (F), semi-quantitative measurements of SOX-9, and (G), quantification of Alcian blue staining (3 weeks). (H to J), Adipogenic differentiation: (H), fluorescence micrographs, (I), semi-quantitative measurements of PPARγ, and (J), quantification of Oil Red 0 staining (3 weeks). Scale bars in panels A, B, E, and H: 500 μm. n=3.

FIGS. 5A-5E provide chick ex ovo culture and CAM assay for investigating the vascularization in cryobioprinted scaffolds. (A), Representative images of chick ex ovo culture with implanted cryobioprinted scaffolds for CAM assay in different timesteps. Scale bars: 2 cm for the low-magnification images and 2 mm for the high-magnification images. (B), Angiogenic abilities of the scaffolds and Goldner's trichrome staining showing erythrocytes in red and collagen in green, where the blood vessels are indicated by yellow arrowheads (control: GelMA+CPA, HUVEC: GelMA+CPA+HUVECs, VEGF: GelMA+CPA+VEGF, H&V: GelMA+CPA+HUVECs+VEGF, and RT: GelMA+CPA+HUVECs+VEGF bioprinted at RT). Scale bars: 2 mm for optical images and 200 μm for histology images. (C to E), Quantification of the BV growth surrounding and within the cryobioprinted constructs by measuring different parameters: (C), lengths of the BVs surrounding the scaffolds, (D), BV densities, and (E), the ratio of BV-to-tissue areas within the scaffolds. n=5; *P<0.05.

FIGS. 6A-6G provide schematic illustrations of vertical 3D cryo(bio)printing and its application in muscular tissue engineering. A) The GelMA-based hydrogel when subjected to directional freezing, forms interconnected gradient and anisotropic microchannels along the vertical axis. B) Vertical 3D cryo(bio)printing of hydrogel filament arrays. C) Vertical 3D cryo(bio)printing of hydrogel filaments of different angles. D) Multi-material vertical 3D cryo(bio)printing of a single hydrogel filament. E) Multi-material vertical 3D cryo(bio)printing of hydrogel filament array. F, G) Vertical 3D cryobioprinting for fabricating (F) the MTU and G) the MMVU.

FIGS. 7A-7K provide graph and image characterizations of the vertically ice-templated 7.5% GelMA constructs. A) Schematic of the vertically ice-templated GelMA construct, and the cross-section showing microchannels perpendicular to the freezing direction. According to the temperature gradient, the construct was sliced into 4 pieces (I, II, III, and IV). B) Fluorescence micrographs showing the effect of temperature gradient on the microchannel sizes in the GelMA (7.5%) construct, pre-labeled with rhodamine B. C) Corresponding quantification results of microchannel diameters at the different temperature layers. D) Schematic showing the microchannels along the freezing direction in the vertically ice-templated GelMA construct. E) Fluorescence micrograph showing a gradient scaffold with microchannel width increasing from bottom (left) to top (right). F) Fluorescence micrographs showing the diameter of the microchannels in the scaffolds decreasing with the freezing-plate temperature changing from −5° C. to −20° C. G) Corresponding quantification results of the diameters of the microchannels. H) Compressive stress—strain curves and I) compressive moduli of the GelMA hydrogels directionally frozen at −20° C. J) Tensile stress—strain curves and K) tensile moduli of GelMA hydrogels directionally frozen at −20° C. The control samples were GelMA hydrogels undergoing no freezing steps (*p<0.05).

FIGS. 8A-8K provide graphs and images showing vertical 3D cryoprinting experimental and simulation results. A) Schematic showing the vertical 3D cryoprinting process. B) Photographs showing changes in the freezing and printing heights in cryoprinted 7.5% GelMA pillars as a function of the freezing plate temperature. C) Corresponding quantification results of the vertical freezing and printing heights. D) Simulated temperature gradient in a vertical structure at 480 s after cryoprinting. E) Simulated height of freezing in the cryoprinted structure gradually increasing with time. F) Simulated temperature distributions throughout the vertically cryoprinted constructs decreases gradually over time. G) Fluorescence photographs showing vertically cryoprinted GelMA bioink arrays containing 4, 9, and 16 pillars. H) Fluorescence photographs showing vertically cryoprinted GelMA constructs at different angles. I) Corresponding quantification results of the printable lengths at different angles. J) Fluorescence photographs showing cryoprinted sophisticated GelMA structures. K) Fluorescence photographs showing vertically cryoprinted 2-by-2 pillar arrays by bioinks without thermogelling properties (3% alginate, 25% PEGDA, and 5% ColMA).

FIGS. 9A-9K provide graphs and images showing vertical 3D cryoprinting of various single- and multi-material GelMA constructs. A) Schematic and fluorescence images showing vertically cryoprinted hollow constructs consisting of different wall thicknesses, fabricated with coaxial nozzles containing varying inner needle sizes. B) Schematic and fluorescence images showing vertically cryoprinted multi-material core-shell structures, fabricated with coaxial nozzles with varying inner needle sizes. C) Schematic and fluorescence images showing vertically cryoprinted multi-material programmable heterogeneous solid structures fabricated, fabricated with a coaxial nozzle. D) Quantification results of the wall thicknesses and the core sizes in vertically cryoprinted hollow structures shown in (A, B). E-G) Fluorescence intensity profiles of vertically cryoprinted heterogeneous solid structures shown in (C). For the lines in the plots, the starting point of each line is 0 and the end point is 1, as indicated in in the lines shown in the corresponding micrographs. H-K) Schematics, photographs, and fluorescence images showing vertically cryoprinted multi-compartmental structures, fabricated with nozzles with varying amounts of bundled channels. For the lines in the plots, the starting point of each line is 0 and the end point is 1, as indicated in the circles shown in the corresponding micrographs (*p<0.05).

FIGS. 10A-10I provide graphs and images showing behavioral characterizations of C2C12 cells encapsulated in the vertically cryobioprinted GelMA constructs. A) Bright-field and fluorescence micrographs showing viability of encapsulated C2C12 cells in the frozen and unfrozen areas in a single vertically cryobioprinted structure on Days 1, 3, and 7 of culture. Live cells were stained in green and dead cells in red. B) Corresponding quantification results of cell viability. C) Fluorescence micrographs showing myotube-formation from Day 1 to Day 7 in the frozen area of a vertically cryobioprinted structure. D) Corresponding quantification results of myotube lengths. E) Fluorescence micrographs showing myotube-formation in the frozen and unfrozen areas on Day 7. F) Confocal reconstruction showing aligned myotubes in the frozen segment on Day 7. G-I) Corresponding quantification results of (G) myotube spreading areas, (H) myotube angle distributions, and (I) myotube lengths on Day 7 (*p<0.05).

FIGS. 11A-11L provide graphs and images showing MTU and MMVU fabricated via vertical 3D cryobioprinting. A) Schematic of the MTU reconstructed by C2C12 myoblasts and NIH/3T3 fibroblasts. B) C2C12 (green) and NIH/3T3 (red) cells labeled with fluorescence tracers and vertically cryobioprinted as an MTU model on Day 7. C) Fluorescence micrographs showing MTU-formation from Day 1 to Day 7 after vertical 3D cryobioprinting. D-F) Quantification results of (D) cell lengths from Day 1 to Day 7, (E) fluorescence intensity profiles on Day 7, and (F) C2C12 and NIH/3T3 spreading angle distributions on Day 7 of the MTU. The starting point of the fluorescence intensity profile was 0 and the end point was 1, which represented the longitudinal MTU interface. G) Schematic of the MMVU reconstructed by C2C12 myoblasts and HUVECs. H) C2C12 cells (green) and HUVECs (red) labeled with fluorescence tracers and vertically cryobioprinted as an MMVU model on Day 7. I) Fluorescence micrographs showing MMVU-formation from Day 1 to Day 7 after vertical 3D cryobioprinting. J-L) Quantification results of (J) cell length from Day 1 to Day 7, K) fluorescence intensity profiles on Day 7 and L) C2C12 and NIH/3T3 spreading angle distribution on Day 7 of the MMVU after vertical 3D bioprinting. The starting point of the fluorescence intensity profile was 0 and the end point was 1, which represented the lateral MMVU interface (*p<0.05).

### DETAILED DESCRIPTION

A method of making a 3D-printed biomaterial is provided. The method includes depositing a hydrogel comprising cells and a cryoprotectant from a 3D printer onto a freezing plate to form a frozen hydrogel filament; depositing additional hydrogel comprising cells and a cryoprotectant from a 3D printer in contact with a previously placed frozen hydrogel filament to form a 3D-printed biomaterial comprising a plurality of frozen hydrogel filaments; and removing the 3D-printed biomaterial from the freezing plate. 3D-printed biomaterials made by the method are also provided.

### Definitions

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. For example, as used in this specification and the appended claims, the singular forms “a”, “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form. The conjunctive phrase “and/or” indicates that either or both of the items referred to can be present.

The phrase “consisting essentially of” means that the composition or method may include additional ingredients and/or steps, but only if the additional ingredients and/or steps do not materially alter the basic and novel characteristics of the claimed composition or method.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾ This applies regardless of the breadth of the range.

As used herein, the term “about” means±10% of the recited value.

As used herein, the terms “printing” and “bioprinting” are used interchangeably, and cover both normal 3D printing and printing that includes cells in the bioink.

A weight percent (wt. %) of a component, unless specifically stated to the contrary, is based on the total weight of the formulation or composition in which the component is included. Furthermore, where the weigh percentage of the polymeric components of a hydrogel are provided, it can be assumed that the remaining weight percentage consists of water, cells, and other minor additives that may be present in the hydrogel unless the percentages of other components are specifically provided.

“Biocompatible” as used herein, refers to the capability of a material to be integrated into a biological system without harming or being rejected by the system. Examples of harm include inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts at toxic concentrations, does not cause prolonged inflammation or irritation, or does not induce more than a basal immune reaction in the host.

The term polymer, as used herein, also refers to polymer precursors that are cross-linked to form the polymer, unless it is specifically indicated that the polymers do not include polymer precursors.

### Methods of Making Three-Dimensional (3D)-Printed Biomaterial

One aspect of the invention provides a method of making a 3D-printed biomaterial, comprising depositing a hydrogel comprising cells and a cryoprotectant from a 3D printer onto a freezing plate to form a frozen hydrogel filament; depositing additional hydrogel comprising cells and a cryoprotectant from a 3D printer in contact with a previously placed frozen hydrogel filament to form a 3D-printed biomaterial comprising a plurality of frozen hydrogel filaments; and removing the 3D-printed biomaterial from the freezing plate.

Three-dimensional printing (also known as additive manufacturing) is a method that takes information of the surface shape of a 3D object, models it using computer-aided design/computer-aided manufacturing (CAD/CAM) software, and then slices that model into multiple layers, creating a stacked cross-sectional version of the 3D object. This information is then fed into a 3D printer capable of fabricating the 3D object using an additive or subtractive manufacturing process for constructing three-dimensional single or multi-layered structures disposed on a substrate. Examples of 3D printing methods include stereolithography, direct ink writing, viscous solution printing, and digital light processing.

Different types of 3D printers can be used to deposit the hydrogel onto the freezing plate. In some embodiments, depositing the hydrogel comprises extruding the hydrogel from, for example, a coaxial 3D printer. In some embodiments, the bioink is applied using an inject printing method (e.g., droplet-based printing) or a vat polymerization printing method, such as stereolithographic printing, digital light processing, or volumetric printing. Levato et al., Nature Reviews Methods Primers, 3, 47 (2023). In some embodiments, microfluidics-enhanced bioprinting can be used. See Chatinier et al., Biomicrofluidics, 15, 041304 (2021). Microfluidic systems contain channels on the micrometer-scale and can facilitate precise positioning of different materials.

In other embodiments, the hydrogel can be used with different types of 3D printing methods. In some embodiments, the hydrogel is applied by being extruded using an extrusion print-head. In other embodiments, the hydrogel is applied using a stereolithographic printing apparatus. In further embodiments, the hydrogel is applied using an inkjet/droplet method. The steps involved in carrying out these methods are known to those skilled in the art.

The 3D-printed biomaterial is typically crosslinked before the material is used. For example, the biomaterial can be crosslinked shortly after the 3D-printed biomaterial has been formed on the freezing plate, or the 3D-printed biomaterial can be crosslinked after being stored.

A variety of methods are available for crosslinking hydrogels, depending in part on the hydrogel being used. In some embodiments, the hydrogel (e.g., alginate) is physically (e.g., ionically) cross-linked by calcium (e.g., CaCl₂) while in other embodiments the hydrogel (e.g., gelatin) is cross-linked using microbial transglutaminase (mTG). In further embodiments, the hydrogel is cross-linked by exposure to ultraviolet or visible light and using a photoinitiator. The wavelength used will generally depend on the photoinitiator used. Examples of suitable wavelengths include from about 200 nm to about 500 nm, from about 250 nm to about 450 nm, or from about 300 nm to about 400 nm.

In some embodiments, the frozen hydrogel filaments are formed in the vertical direction. Vertical, as used herein, refers to perpendicular to the freeze plate. The hydrogel filaments are formed by depositing hydrogel on the freeze plate, followed by further deposition of hydrogel on top of the hydrogel that has already been deposited, forming a column in the vertical direction. This can occur as a result of continuous extrusion of hydrogel onto a specific point of the freeze plate, while gradually raising the extrusion nozzle, or as a result of placing specific droplets of material, first on the freeze plate, and then on previously placed droplets.

The method of preparing a 3D-printed biomaterial includes depositing a hydrogel from a 3D printer onto a freezing plate to form a frozen hydrogel filament. A freezing plate is a 3D printer print plate that has been modified so that it is chilled to a temperature that will freeze the hydrogel when the hydrogel is deposited onto the plate (or onto hydrogel previously deposited on the plate). The freezing plate can be chilled by placing it in proximity to a cold substance such as dry ice or liquid nitrogen, or using a refrigeration technique such as the use of a thermoelectric cooler (i.e., a solid-state heat pump). In some embodiments, the freezing plate is chilled to a temperature ranging from about 0° C. to about −200° C., while in further embodiments the freezing plate is chilled to a temperature ranging from about −5° C. to about −100° C., or from about −5° C. to about −30° C.

In addition to using a freezing plate, there are other methods that can be used to chill the hydrogel. In some embodiments the hydrogel is chilled (i.e., pre-chilled) to a temperature ranging from about 0° C. to about 10° C. before being deposited by the 3D printer, so that its temperature is reduced even before it contacts the freezing plate. Alternately, or in addition, it can also be useful to chill the compartment in which the 3D-printed biomaterial is formed. For example, air chilled to below 0° C. can be blown into the chamber and over the freezing plate to contact the hydrogel as it is deposited. For example, co-extruded liquid nitrogen can be used together with, or as an alternative to the use of a freezing plate to freeze deposited hydrogel.

The hydrogel used to form the 3D-printed biomaterial includes a cryoprotectant. Cryoprotectants are compounds that protect the cells from the freezing temperatures used during formation and storage of the 3D-printed biomaterial. Cryoprotectants include dimethyl sulfoxide (DMSO), glycerol, ethylene glycol, and various saccharides. In particular, disaccharides and trisaccharides can be used as cryoprotectants. Examples of suitable disaccharides include D-(+)-trehalose, D-lactose, and sucrose, while examples of trisaccharides include D-(+)-raffinose and D-(+)-melezitose. In some embodiments, the cryoprotectant is D-(+)-melezitose. The concentration of the cryoprotectant can range from about 1% v/v to about 10% v/v.

The method can be used to create 3D-printed biomaterials having a variety of different shapes. In some embodiments, the deposited hydrogel comprises a scaffold, while in other embodiments the extruded hydrogel comprises a tissue construct such as a blood vessel. Different methods of 3D-printing can be chosen that are better suited to providing the specific shape of interest.

In some embodiments, a plurality of frozen 3D-printed biomaterials are separately prepared and then combined to form a larger structure. This can be done to create shapes that might be difficult to prepare directly on a 3D printer, and/or to combine 3D-printed biomaterials made separately and having different characteristics, such as different porosity and/or different polymer composition. Different blocks (or segments) can be joined, for example, by placing frozen blocks adjacent to one another, allowing them to melt slightly, and then crosslinking the blocks so that they join together.

The 3D-printed biomaterial can be stored before use. To preserve the biomaterial, and cells embedded in the biomaterial, it is preferable to chill the 3D-printed biomaterial during storage. the 3D-printed biomaterial is stored at 0° C. or less. In further embodiments, the 3D-printed biomaterial is stored at a temperature from −20° C. to about −80° C. These temperatures can be achieved by, for example, storing the 3D-printed biomaterial in liquid nitrogen.

The method includes depositing a hydrogel comprising cells and a cryoprotectant. Viable cells that can be included in a 3D-printed object include prokaryotic and eukaryotic cells. Non-limiting examples of eukaryotic cells include mammalian cells such as myoblasts, fibroblasts, endothelial cells, and stem cells. Other living cells include embryonic stem cells, amniotic fluid stem cells, cartilage cells, bone cells, muscle cells, skin cells, pancreatic cells, kidney cells, nerve cells, liver cells, and the like. Viable cells are living cells.

Standard cell culture techniques are typically used when handling the cells for the 3D-printed biomaterial. For example, a portion of or the entire printed article can be placed under standard cell culture conditions (e.g., temperature, pressure, nutrient concentrations, etc.) in order for the cells to remain viable. The 3D-printed biomaterial can comprise from about 1×10¹ to about 1×10⁹ viable cells, or from about 1×10² to about 1×10⁸ viable cells, or from about 1×10³ to about 1×10⁷ viable cells, or from about 1×10⁴ to about 1×10⁷ viable cells, or from about 1×10⁵ to about 1×10⁷ viable cells (all being cells per milliliter).

The hydrogel composition can also include serum to support the cells. A wide variety of serums for supporting cells (i.e., cell culture media) are known, and can vary depending on the type of cells. Examples of suitable serum include fetal bovine serum (FBS) and Dulbecco's modified Eagle medium (DMEM), as well as chicken, caprine, equine, human, ovine, porcine, and rabbit serum. Typically the hydrogel composition includes from about 1% to about 10% v/v of serum.

### 3D-Printed Biomaterials

Another aspect of the invention provides a 3D-printed biomaterial. The biomaterial is made by depositing a hydrogel comprising cells and a cryoprotectant from a 3D printer onto a freezing plate to form a frozen hydrogel filament; depositing additional hydrogel comprising cells and a cryoprotectant from a 3D printer in contact with a previously placed frozen hydrogel filament to form a 3D-printed biomaterial comprising a plurality of frozen hydrogel filaments; and removing the 3D-printed biomaterial from the freezing plate. When initially prepared, the 3D-printed biomaterial is frozen, but it can later be thawed before use. However, in other embodiments, the 3D-printed biomaterial is kept frozen, for example when the biomaterial is being kept in storage before use.

Because of their biocompatibility, hydrogels are preferred for use in bioink formulations, since hydrogels are water-rich polymers that can hold considerable amounts of water and are benign to embedded cells. Hydrogels are polymeric networks with hydrophilic chains crosslinked either covalently or physically (via intra- and intermolecular attractions). Biocompatible hydrogels include natural hydrogels and synthetic hydrogels. Examples of natural hydrogels include hyaluronic acid, hyaluronic acid derivatives (e.g., hyaluronic acid methacrylate), silk (e.g., silk fibroin or silk sericin), collagen, gelatin, gelatin derivatives (e.g., gelatin methacryloyl) and alginate. Examples of synthetic hydrogels include polyethylene glycol derivatives and polyvinyl alcohol derivatives. Hydrogels can also include natural hydrogels that have been modified, such as methacrylated collagen. In some embodiments, the 3D-printed biomaterial comprises a plurality of different hydrogels.

One of the polymers that can be included in the hydrogel is gelatin methacryloyl (GelMA), also known as gelatin methacrylate or gelatin methacrylamide, is typically prepared by reaction of gelatin with methacrylic anhydride. Sun et al., Polymers (Basel)., 10(11): 1290 (2018). A variety of different concentrations of GelMA can be used. In some embodiments, the GelMA has a concentration ranging from 1% to 3% by weight, 2% to 4% by weight, 3% to 5% by weight, 1% to 5% by weight, 2.5% to 7.5% by weight, 5% to 10% by weight, 7.5% to 15% by weight, 10% to 15% by weight, 10% to 20% by weight, 15% to 20% by weight, or from 15% to 25% by weight.

Another polymer that can be included in the hydrogel is alginate. Alginate is a natural anionic polymer typically obtained from brown seaweed consisting of linear copolymers of β-(1-4) linked d-mannuronic acid (M) and β-(1-4)-linked 1-guluronic acid (G) units. In some embodiments, the alginate has a concentration ranging from 0.1% to 1.5% alginate, from 0.1% to 3% alginate, from 0.5% to 2.5% alginate, from 1% to 3% alginate, or from 1% to 5% alginate, all by weight. In some embodiments, the alginate has a molecular weight from about 100 kDa to about 200 kDa, from about 200 kDa to about 300 kDa, or about 100 kDa. In some embodiments, the alginate has an M to G ratio (M/G) from about 0.3 to about 0.7, from about 0.7 to about 1.0, or from about 1.0 to about 1.3.

In some embodiments, the 3D-printed biomaterial comprises microchannels. While not intending to be bound by theory, the inventors proposed that interconnected microchannels are formed when ice crystals formed during the freezing process melt. The microchannels can have a size ranging from about 10 μm to about 1000 μm, or in some embodiments, from about 50 μm to about 600 μm. In further embodiments, the microchannels form an anisotropic gradient. An anisotropic structure is one that has a different value when measured in different directions. For example, anisotropic 3D-printed biomaterial can include microchannels having a diameter that differs when measured at different points along the vertical direction, or when measured along the vertical direction versus the horizontal direction. In one embodiment, the microchannels along the bottom of the biomaterial have a diameter from about 50 μm to about 100 μm, while the microchannels along the top of the biomaterial have a diameter from about 400 μm to about 600 μm.

The 3D-printed biomaterials can have essentially any size and shape that can be obtained using a 3D-printer. In some embodiments, the 3D-printed biomaterial is shaped as an object intended for medical or pharmaceutical use, such as a tissue scaffold (e.g., an artificial transplant support). In some embodiments, the 3D biomaterial is a soft tissue construct (e.g., an organoid). In some cases, the 3D biomaterial can be personalized for a specific subject by basing the 3D object on an image obtained from magnetic resonance imaging, computed tomography, or ultrasound. A wide variety of tissue engineering applications for 3D-printed biomaterials comprising hydrogels are known to those skilled in the art. Advincula et al., MRS Commun., 11(5):539-553 (2021).

In some embodiments, the 3D-printed biomaterial is a tissue construct. An example of a 3D-printed biomaterial tissue construct is neo-cartilage. In some embodiments, the 3D-printed biomaterial can be a soft tissue construct. Soft tissues connect and support other tissues and surround the organs in the body. They include muscles (e.g., the heart), fat, blood vessels, nerves, tendons, and tissues that surround the bones and joints. Examples of 3D soft tissue constructs include skin, musculoskeletal tissue, cardiac tissue, heart valve, liver, and neuronal tissue. In some embodiments, the 3D-printed biomaterial is a muscle tissue scaffold. The cells included in the tissue construct are preferably the type of cells normally found in the particular type of tissue, or precursor cells (e.g., stem cells) that will result in that particular type of tissue. The cells may be substantially uniformly distributed throughout the polymer, or they may be suspended within a part of the polymer. Examples of suitable cells include myoblasts, fibroblasts, endothelial cells, or stem cells.

In some embodiments, the 3D-printed biomaterial can be provided in the shape of an organ or tissue, such as circulatory organs. In some embodiments, the biomaterial comprises a hollow tube. A hollow tube can be used as a blood vessel, such as an artery or vein. The hollow tube comprises a cylinder having a hollow interior, an inner side facing the hollow interior, and an outer size facing the exterior of the cylinder. Blood vessels can have a variety of sizes, depending on the type of blood vessel, with a normal human aorta having a diameter of about 2 cm, while capillaries can have a diameter from about 2 to 12 μm. Accordingly, the blood vessels can have a diameter from about 2 μm to 2 cm, with non-capillary blood vessels having a diameter ranging from about 1 mm to about 2 cm.

The 3D-printed biomaterial can also include one or more additives. Non-limiting exemplary additives for the biomaterial include diluent synthetic polymers (e.g., polyethylene glycol, polypropylene glycol, poly(vinyl alcohol), poly(methacrylic acid)), drugs (e.g., antibiotics such as penicillin and streptomycin), cell nutrients (e.g., proteins, peptides, amino acids, vitamins, carbohydrates (e.g., starches, celluloses, glycogen), and minerals (e.g., calcium, magnesium, iron), synthetic or naturally occurring nucleic acids, absorbers to limit light penetration, inhibitors (e.g., scavengers and quenchers), refractive index modifiers (e.g., iodixanol), and nanocomposite components such as graphene or silica. The bioink formulation can comprise one or more additives in an amount of 0 wt % to about 25 wt % of the composition, based on total weight of the composition.

The present invention is illustrated by the following examples. It is to be understood that the particular examples, materials, amounts, and procedures are to be interpreted broadly in accordance with the scope and spirit of the invention as set forth herein.

### EXAMPLES

### Example 1: Freeform Cell-Laden Cryobioprinting for Shelf-Ready Tissue Fabrication and Storage

Here, we report an unconventional technology, termed cryobioprinting, that exploits 3D cell-laden bioprinting and cryopreservation methods to concomitantly fabricate and store bioprinted tissues for on-demand applications. By using this embodiment, the products are stored for a long(er)-term period and used after crosslinking, thawing, and resuscitating. The idea of cryogenic 3D printing of acellular inks has been recently proposed (Wang Z, and Florczyk SJ, Materials. 13, 354). However, the incorporation of living cells within the bioink and bioprinting under cryogenic conditions, i.e., cryobioprinting as we define, remains very limited and requires many more considerations to ensure the efficacy of the method.

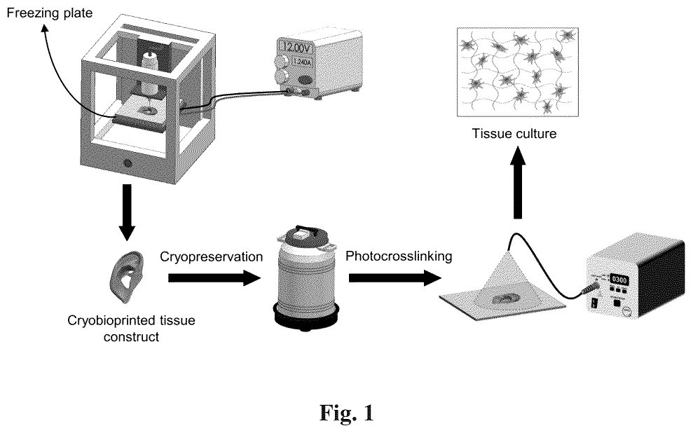

As schematically briefed in FIG. 1, a cell-laden hydrogel precursor (i.e., the bioink) is used for bioprinting tissue constructs on a customized freezing plate. The frozen bioprinted samples are immediately further cryopreserved for short- and long-term storage. When needed, the cryopreserved tissue constructs can be readily transported under cryogenic conditions, crosslinked, and revived on-site for subsequent usage. Gelatin methacryloyl (GelMA) (Gong J. et al., Nat. Commun 11, 1-14) and various CPAs are employed to design the optimum bioink. We investigate the effect of bioink composition (GelMA, DMSO, and one of the six types of saccharides) on the cryopreservation and resuscitation of various cells. The optimized bioink configuration is selected based on the cell viability results. A range of two-dimensional (2D) patterns and 3D structures are cryobioprinted with the optimized bioink to confirm the bioprinting performance. After resuscitating the samples, the functionality and stemness of the encapsulated human mesenchymal stem cells (hMSCs) are verified via osteogenic, adipogenic, and chondrogenic differentiations. Furthermore, the angiogenesis potential of the cryobioprinted constructs is assessed using the ex ovo chorioallantoic membrane (CAM) assay. Our results show that cryobioprinting provides a high-fidelity strategy to fabricate shelf-ready cell-laden constructs that can be broadly used in applications including but not limited to tissue engineering, regenerative medicine, tissue model engineering, and drug screening.

### Results

### Freezing Plate

A thermoelectric cooler, which is a solid-state heat pump and works based on the Peltier effect (Kishore RA, et al., Nat. Commun 10, 1-13), was designed and fabricated to maintain tightly controlled low temperatures on the substrate surface. This adjustable device transfers the heat from the printing stage, generating a precisely controllable temperature drop. The freezing plate temperature was adjusted via changing the input voltage or current. The two tables also suggest that the effect of environmental temperature on the freezing plate's temperature was negligible. Upon extrusion from the bioprinter nozzle and contact with the freezing plate, the bioink rapidly solidifies in situ and forms a stable structure.

### Printability and Fidelity

Printability of potential cryoprotective bioinks with different DMSO concentrations was evaluated. Living cells were not used at this step, as the presence of the cells was assumed not to significantly affect the results of interest. The effect of parameters such as the pressure, the printhead moving speed, and the nozzle size was examined by cryobioprinting a grid structure using 5% (w/v) GelMA containing different DMSO concentrations. We used a 27G nozzle for cryobioprinting jobs since it generally yielded a better printing quality than a 30G nozzle. A lower printhead moving speed improved spatial resolution within the range of speed and pressure values investigated (quantitatively analyzed. As expected, increased pressure while keeping printhead speed constant at 10 mm s⁻¹ caused a considerable enlargement in the filament diameter.

The cryobioprinted structures were either immediately crosslinked or cryopreserved in a liquid nitrogen tank for on-demand use. The ultraviolet (UV) exposure times needed for crosslinking 4-layer, 8×8-mm² GelMA grid patterns with different DMSO concentrations were studied. The samples that were not exposed to UV fully dissolved in phosphate-buffered saline (PBS) after shaking overnight. 10 s of UV exposure resulted in a partially crosslinked sample, which did not maintain its integrity after shaking. Samples that underwent more prolonged UV exposure exhibited an acceptable fidelity, regardless of the DMSO concentration. The minimum essential UV exposure time, i.e., 20 s, was subsequently used for crosslinking the cell-laden grid structures without compromising cell viability. This minimal UV exposure is deemed to be safe for the living cells (Mironi-Harpaz I, et al., Acta Biomater. 8, 1838-1848). In addition, an identical crosslinking regime was used for all the samples, including the control, during the sample-preparation for biological tests. As a result, the effect of crosslinking method, even if minimal, was omitted from the analysis.

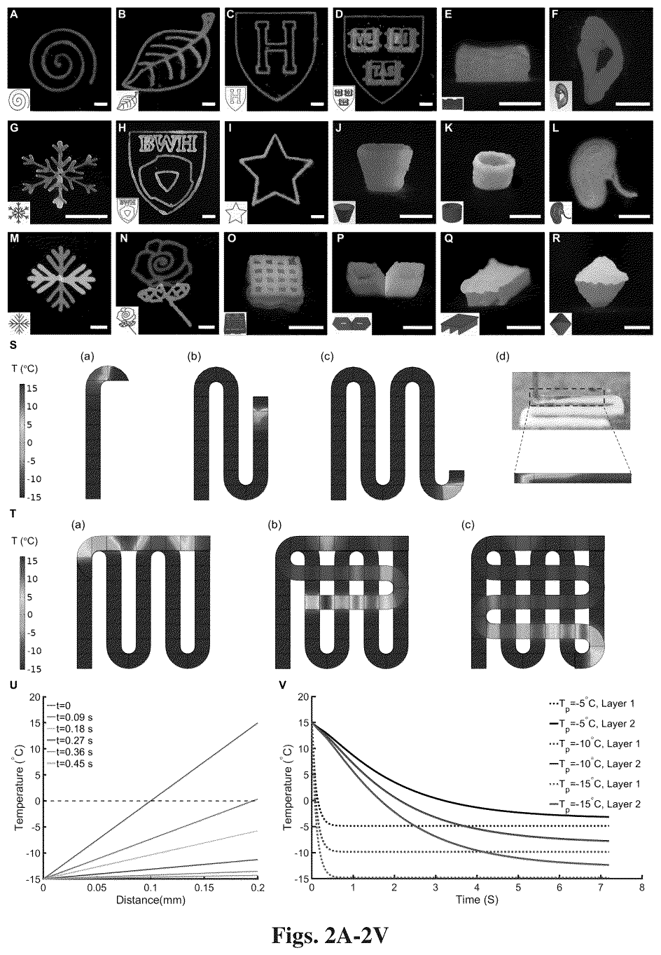

UV crosslinking was the first step after taking out the samples from the liquid nitrogen tank or other cryopreservation conditions. The samples were then rapidly thawed in pre-warmed PBS at 37° C. to avoid ice crystal-formation and simultaneously remove the residual CPAs. No noticeable damage, such as visible cracks, was observed after thawing. Representative cryobioprinted structures from simple 2D patterns to sophisticated 3D constructs were successfully created from GelMA- and gelatin-based bioinks (FIG. 2, A to R). Multi-material 2D and 3D constructs were subsequently fabricated from GelMA and gelatin, as shown in FIG. 2, M to R. Remarkably, we used 5% (w/v) GelMA or gelatin as the base bioink to create freeform constructs (FIG. 2, P to R), some of which could be complicated via conventional direct extrusion bioprinting methods.

### Heat-Transfer Simulation

The layer-by-layer cryobioprinting of a grid structure was modeled in COMSOL Multiphysics® to understand the mechanism of heat-transfer during the process. The simulation of heat-transfer in the first layer while it is being cryobioprinted was recorded. Since the first layer directly contacts the freezing plate (−15° C.), the heat-transfer from the bioink to the freezing plate occurs instantaneously. Once the bioink contacts the freezing plate, the frozen region expands through the deposited material until the entire layer is frozen. Experimentally, the frontier of the frozen bioink perfectly matched that of the simulated model, indicating a good agreement between the simulation and the experiment. The freezing regime in the second layer is slower since the entire layer is not touching the underlying frozen layer, as shown in FIG. 2T.

Temperature change through the first layer's filament thickness was plotted for different time steps to analyze the cryobioprinting procedure quantitatively (FIG. 2U). At t=0, the first layer's bottom surface is exposed to the freezing plate temperature, while the top of the layer remains at 15° C. Since the heat conduction in the bioink obeys Fourier's law, the temperature through the thickness changes linearly with the distance under a constant temperature gradient. At t=0.09 s, the whole bioink is frozen, and the top surface temperature reaches approximately 0° C. This trend continues until the entire simulated section reaches an isothermal steady state at t=0.45 s. In FIG. 2V, the average surface temperatures of the layers are compared for three different freezing plate temperatures (Tₚ). For Tₚ=−15° C., the top surface temperature of the second layer reaches 0° C., after 1.7 s, which corresponds to the freezing point of the bioink. Increasing Tₚ to −10° C. and −5° C. results in a slower freezing regime in the second layer, while the rapid temperature change in the first layer is not remarkably affected. As expected, the time needed to completely freeze the second layer is longer than that of the first layer for all three Tₚ values.

### Ice Crystal-Formation

One critical factor that affects cell viability during cryopreservation is the formation of ice crystals (Cheng Y, et al., Small 15, 1904290). The spicular ice crystals can damage the cell membrane, resulting in lower cell viability. Real-time recordings of the ice crystal-formation in GelMA hydrogel groups with different CPA concentrations shows the corresponding time-lapse images. The addition of CPAs noticeably affected the shape of ice crystals. In the control sample (without DMSO), the ice crystals were more spicular and formed faster than the other study groups. Increasing the DMSO concentration led to the formation of fine ice crystals; however, no remarkable improvement in ice-crystallization was observed when the saccharide concentration was increased, as the saccharide merely prevents osmotic damage to the cells. The fractal dimension (FD) theory, in conjunction with an image processing method, was employed to quantitatively compare the smoothness and the growth rate of the ice crystals. The FD value quantifies the geometry's irregularities. In our analysis, ice crystals' boundaries were extracted from the brightfield images and digitized. The FD value for the resulting quasi-circle geometry was then calculated using equation (1) (Chen Y, Math. Probl. Eng 2020, 7528703).

$$F D = \frac{2 ln ( P / 2 π ) )}{ln ( A / π )} \qquad ( 1 )$$

, where P and A stand for the perimeter and the area of the quasi-circle geometry, respectively. FD is an indicator of how the quasi-circle geometry deviates from a circle. When the geometry of interest is a perfect circle, the FD equals 1. As the irregularities and sharp edges appear in the geometry, the FD increases. For the freezing bioink, when ice crystals form, the geometry of the unfrozen region starts to diverge from a perfect circle, leading to an increased FD. The trends of changes in FD for freezing bioinks with different DMSO concentrations are documented. In the control sample, i.e., DMSO-free, the highly spicular ice crystals formed rapidly, and therefore, the FD value was higher than those of the other groups. When 90% of the field of view was frozen, the FD values were recorded and compared. It is evident that the addition of DMSO significantly decreased the FD value. The percentages of the frozen areas were plotted over time to compare the freezing rates. The control sample froze faster than the other samples, and as the DMSO concentration increased, the bioink started to freeze slower.

### Splat Assay

To further confirm the effect of CPAs on inhibiting ice-recrystallization, a modified splat assay was employed. Cryomicrographs of annealed ice wafers clearly demonstrated that ice crystals were formed during the freezing of GelMA. The results showed that the average size of the ice crystals significantly decreased when DMSO concentration was increased. Smaller crystal size is an indication of enhanced ice recrystallization-inhibition (Tomczak MM, et al., Biochem. Biophys. Res. Commun 311, 1041-1046).

### Cryoprotective Bioink Design Based on Cell Viability Assays

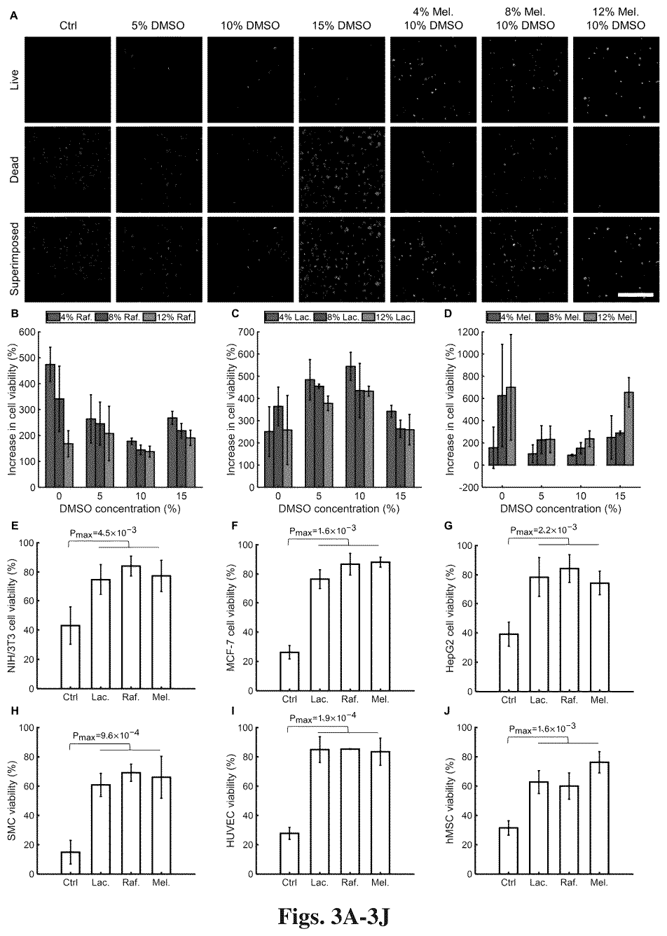

A series of cell viability experiments were conducted using different concentrations of DMSO and saccharides to find the optimized configuration of CPAs in GelMA. Since monosaccharides generally show less protective effects regardless of concentration and freezing trend (An TZ, et al., Cryobiology. 40, 237-249), we focused on using disaccharides (lactose, maltose, sucrose, trehalose) and trisaccharides (raffinose, melezitose) along with DMSO as the CPAs. The cell-laden bioink was used to produce samples containing different concentrations of DMSO and saccharides at room temperature (RT). The cast samples were then directly transferred to −80° C. for 24 h, conventionally cryopreserved in liquid nitrogen for 72 h, crosslinked via UV exposure, and underwent live/dead assays. FIG. 3A shows a representative set of live/dead images for immortalized mouse myoblast cells (C2C12) cryopreserved in 5% (w/v) GelMA with different DMSO and melezitose concentrations. By using the fluorescence images, the cell viability ratios of C2C12 cells were determined. Regardless of the DMSO concentration, the saccharide-free groups generally showed a lower cell viability rate since the presence of saccharides avoids osmotic shock during the thawing process (Solocinski J, et al., Cryobiology. 75, 134-143). It is, therefore, concluded that the saccharide is an essential part of a cryoprotective bioink.

Cell viability of each group was normalized to the control group with the same DMSO concentration to choose the most functional saccharide and its optimum concentration (FIG. 3, B to D). The most effective saccharides that led to a higher cell viability increase were 12% (w/v) melezitose, 4% (w/v) raffinose, and 4% (w/v) lactose. The increase in cell viability of the groups with respect to the DMSO-free group with the same saccharide concentration was calculated to identify the most effective DMSO concentration. The optimum concentration of DMSO was 10% (v/v) for the selected sugar types. As a result, three combinations of CPAs were selected to be used with 5% (w/v) GelMA for the next set of experiments: 12% (w/v) melezitose+10% (v/v) DMSO, 4% (w/v) lactose+10% (v/v) DMSO, and 4% (w/v) raffinose+10% (v/v) DMSO. In the following sections, we refer to these selected groups using the names of their saccharides only, considering that they all contained 10% (v/v) DMSO and 5% (w/v) GelMA.

### Using Different Cell Types to Assess the Effectiveness of the Selected CPAs

The efficacy of the CPAs varies with the cryopreserved cell type and cell size (Smith GD and Takayama S, Reprod. Fertil. Dev. 31, 93-104). We studied the cell viability of six other cell types, including mouse embryonic fibroblast cell line (NIH/3T3), human liver cancer cell line (HepG2), human breast cancer cell line (MCF-7), primary human smooth muscle cells (SMCs), primary human umbilical vein endothelial cells (HUVECs), and primary hMSCs, using the three selected bioink groups to assess the feasibility of our method. As demonstrated in FIG. 3, E to J, all the cell types were significantly more viable after 72 h of cryopreservation in the selected groups comparing to the control group, i.e., the group without any CPAs. The high cell viability values for all the cell types confirmed the effectiveness of all three CPA combinations. Given the cell viability rates, the melezitose group was selected for subsequent cryobioprinting jobs, as it yielded a cell viability rate of —80% for the investigated cell types. The fluorescence images of the studied cell types are presented.

Effect of the CPAs on the Cells before Cryobioprinting

In extrusion bioprinting, it is inevitable to keep the cells in contact with the bioink/CPAs for several minutes if not longer. Contact with the CPAs over an extended time period before cryobioprinting may jeopardize cell viability due to toxicity of the DMSO and the prolonged exposure to osmotic pressure caused by the presence of saccharide molecules. We quantified the cell viability of the NIH/3T3 fibroblasts encapsulated in GelMA/CPA after being kept at RT for different time periods. It was shown that increased exposure time yields lower cell viability. We, therefore, minimized exposure time for our cryobioprinting jobs by using small aliquots of bioinks for each round. The viability drop within 60 min of exposure was still quite acceptable at >80%.

### Effect of Freezing Rate on Cell Viability

The sample freezing rate during the cryobioprinting process has a critical impact on ice crystal-formation and cell viability. Different freezing rates were achieved by changing the T. Given the dynamic nature of cryobioprinting, a constant freezing rate cannot be achieved throughout the construct. The average freezing rates in the first two layers of the cryobioprinted grid structure using different values of Tₚ were presented. As expected, the freezing rate is higher when a lower temperature is used for the freezing plate. Since the second layer does not directly touch the freezing plate, the freezing rate is remarkably lower comparing to the first layer. As a result, the top layers generally have a moderate freezing rate, closer to what is used in conventional cryopreservation (i.e., ˜1° C. min⁻¹). Representative live/dead images of the NIH/3T3 fibroblasts cryobioprinted on the freezing plate with various temperatures, −5° C., −10° C., −15° C., and −20° C. The samples were stained and imaged immediately after cryobioprinting. For cases where Tₚ was −15° C., the cell viability was not significantly different from the original unbioprinted bioink. However, decreasing the Tₚ to −20° C. caused a major decline in cell viability (only ˜60% viable cells).

### Cell Viability in Different Layers

Since the freezing regime is not homogenous throughout the thickness (FIG. 2, T and V), we investigated the cell viability of C2C12 myoblasts in different layers of the cryobioprinted constructs. Three representative layers with an average distance of 3 mm, termed upper, middle, and lower layers, were imaged on days 1, 3, and 7 after cryobioprinting. The cell viability was not noticeably affected by the distance from the freezing plate. The average cell viability within different layers of the scaffold was all about 85%, which is in an acceptable range.

### Medium-Term and Long-Term Cryopreservation

Cryobioprinted hMSC-laden samples were cryopreserved for 14 and 28 days to study the effect of medium-term cryopreservation on cell viability. No significant difference between the cell viability rates of the cryopreserved samples in liquid nitrogen was observed after day 14 and day 28. More excitingly, the viability of NIH/3T3 fibroblasts and HUVECs were further investigated after 3 months of cryopreservation and determined to be 83% and 86% after 7 days of culture, respectively, proving the efficacy of our cryobioprinting method for longer-term storage.

### Cell Differentiation

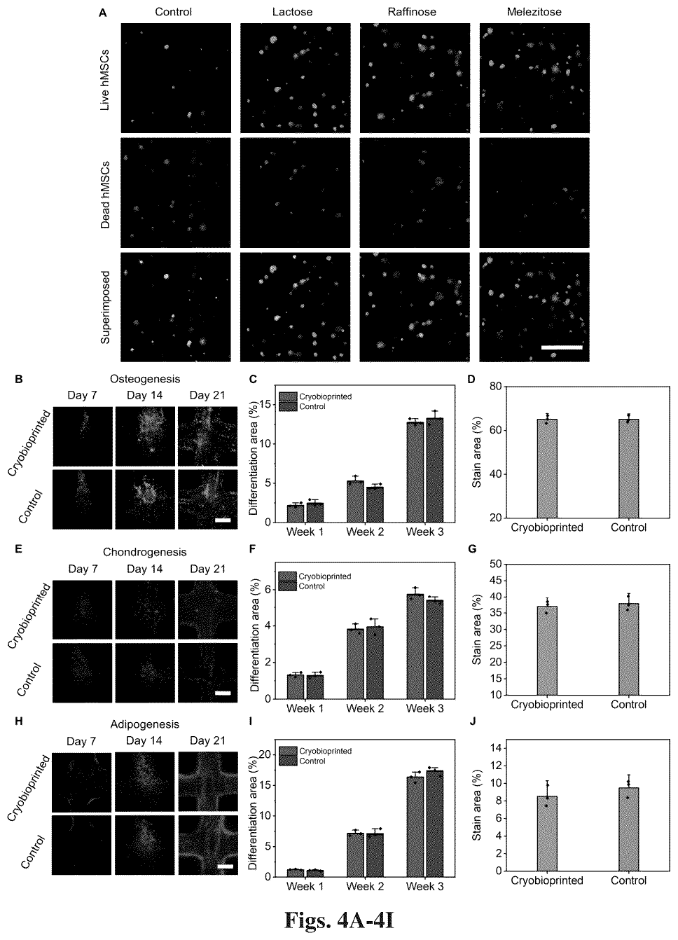

We used hMSCs to investigate their functionality through cell differentiation assays after cryobioprinting. The representative live/dead images for hMSCs after 72 h of cryopreservation are shown in FIG. 4A. Based on our medium-term results, we used cryobioprinted hMSC-laden samples after 14 days of cryopreservation in liquid nitrogen. On day 14, the samples were crosslinked, resuscitated, and cultured for 1 week before adding the differentiation media. The three types of cell differentiation, including osteogenic, chondrogenic, and adipogenic, were studied. In osteogenesis differentiation, runt-related transcription factor 2 (RUNX2, 1 week) and osteocalcin (2 and 3 weeks) immunostaining were conducted. Peroxisome proliferator-activated receptor gamma (PPARγ) and SRY-Box transcription factor-9 (SOX-9) immunostaining assays were used for assessing adipogenic and chondrogenic differentiation, respectively. In FIG. 4, B, E, and H, the fluorescence and immunostaining images of the encapsulated hMSCs after being cultured in differentiation media for 7 days, 14 days, and 21 days are presented. The quantitative analyses of osteogenesis (FIG. 4, C and D), chondrogenesis (FIG. 4, F and G), and adipogenesis (FIG. 4, I and J) revealed that the trends of differentiation in cryobioprinted samples were not notably different than those in control samples (i.e., the unfrozen samples). This finding proves that cryobioprinting did not negatively affect the functionality of hMSCs.

### CAM Assay

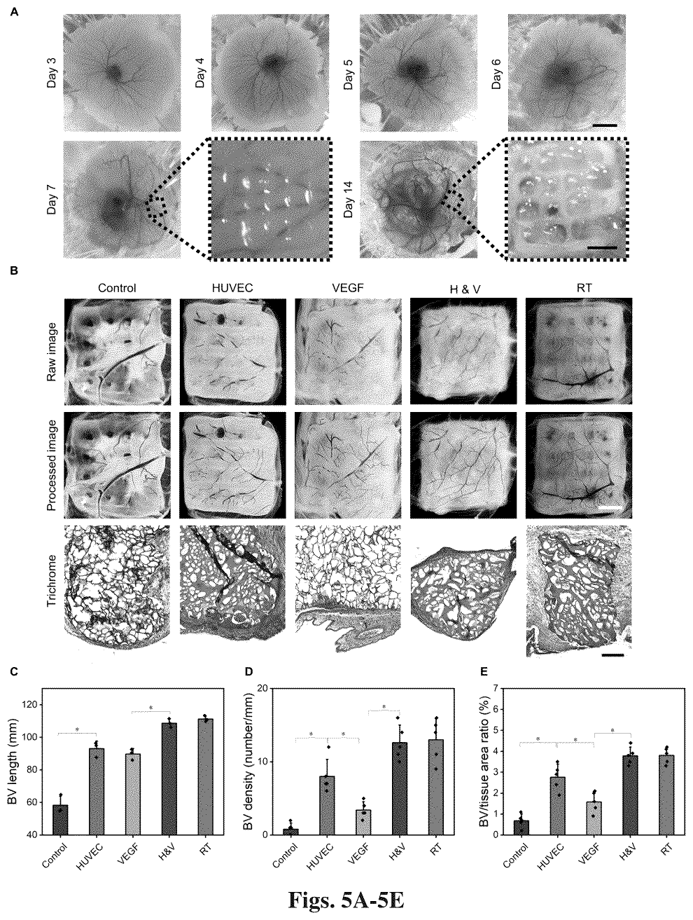

CAM assay was used to evaluate angiogenesis potentials of the cryobioprinted scaffolds. The cryobioprinted constructs (previously stored in liquid nitrogen for 14 days) were implanted on day-7 ex ovo CAM and were collected after 7 days of additional incubation (FIG. 5A). The extents of angiogenesis in the cryobioprinted groups were determined using an image-processing method and histology (FIG. 5B). The average lengths of newly formed blood vessels (BVs) were measured to quantify the angiogenic responses surrounding the different cryobioprinted constructs. As presented in FIG. 5C, the smallest average length of BVs was associated with the control group (i.e., cryobioprinted GelMA+CPA) while adding either HUVECs or vascular endothelial growth factor (VEGF) resulted in a significant increase in the BV length. Cryobioprinted constructs with both HUVEC+VEGF (H&V) yielded the highest vascular growth. When the same bioink (H&V) was bioprinted at RT without freezing, similar vascular growth was achieved. These observations proved that the cryobioprinting procedure did not affect the functionality of HUVECs and VEGF.

Histological analyses further confirmed that the presence of HUVECs had a significant effect on CAM vessel formation within the 3D constructs (FIG. 5B). Almost no BVs grew into the control group, while the vascular infiltration in the HUVEC group was significantly higher. Similarly, vascular invasion in the H&V group was substantially higher than that of the VEGF group. Furthermore, neovascularization was observed deep in the H&V group, as shown in the histology images.

We also quantified the BV density (FIG. 5D) and the ratio of BV-to-tissue areas (FIG. 5E) within the constructs. BV density was calculated as the number of BVs within 1 mm of the interface along the scaffold/tissue border. The ratio of BV-to-tissue area was defined as the percentage of the BVs area over the area of the section (Choi SW, et al., Adv. Healthc. Mater 2, 145-154). The density of the BVs in the HUVEC group was significantly higher than that in the control group. Similarly, the H&V group showed a significantly higher BV density than the VEGF group. The ratio of BV-to-tissue areas also resulted in a similar trend.

### Discussion

Considering the propensity to use shelf-ready products for clinical and research needs, the development of biofabrication methods capable of creating storable tissue constructs is of paramount importance. In this study, we invented and optimized cryobioprinting, a biofabrication process to simultaneously create and store cell-laden tissue constructs. Our results emphasized the unique synergy between bioprinting and cryopreservation, leading to faithful fabrication of tissue constructs with a decent cell viability rate. One of the key points in our approach was designing a freezing plate that allowed precisely controllable and stable temperature during the cryobioprinting procedure.

The limited printability window of soft materials, especially hydrogels, restricts a narrow boundary for choosing bioinks (Li J, et al., Mater. Sci. Eng. R Rep 140). For example, 5% (w/v) GelMA bioink should be kept at approximately 15° C. or lower to achieve successfully bioprinted constructs (Ying G, et al., Adv. Funct. Mater 30, 2003740). In cryobioprinting, the deposited layers immediately freeze in situ. This rapid solidification enhances the printability of the bioink and fidelity of the 3D-bioprinted constructs, providing an expanded range of options to be used as bioinks. Free-standing and oblique constructs have been previously bioprinted using a cell-friendly supporting bath as the collecting substrate (Bao G, et al., Mater. Horizons 7, 2336-2347). Our cryobioprinting approach offers another suitable method for bioprinting such constructs (FIG. 2, P to R), without any need for supporting baths, extra supports, or sacrificial materials. Another advantage of cryobioprinting is the capability of fabricating ready-to-use products. No washing steps are required to remove excessive supporting materials that exist in embedded bioprinting. This feature can transform cryobioprinting into a promising method for leveraging the scalability of the 3D bioprinting technology.

Recently, cylindrical hollow struts have been bioprinted in cryogenic conditions by implementing a core-shell nozzle (Lee JY and Kim GH, J Ind Eng Chem. 85, 249-257). While elegant, the presented method is relegated to fabricating fairly simple structures and is not applicable to bioprinted scaffolds with homogeneously dispersed cells. There are also intrinsic limitations associated with cryobioprinting of thick constructs using a freezing plate in our strategy. Although we were able to successfully fabricate relatively thick models, such as the ear model shown in FIG. 2F, printing beyond a certain thickness may slow down the heat-transfer from the top layers. Slower heat-transfer can negatively affect the printing fidelity of bioinks during the cryobioprinting procedure. Lowering the freezing plate temperature can potentially compensate for the decreased heat-transfer rate but may, on the other hand, compromise cell viability, as demonstrated. In addition, the long-term exposure of living cells to CPAs can be detrimental, as mentioned in the Results section. This issue can be addressed by using microfluidic systems (du Chatinier DN, et al., Biomicrofluidics. 15, 041304) for delivering the CPAs to bioink immediately before cryobioprinting. Additional optimizations are needed to investigate possible solutions for addressing these limitations and improving the technology.

In conventional cell cryopreservation, fetal bovine serum (FBS) is normally used as the CPA in place of saccharides. However, since the deviation of FBS contents might affect the cryopreservation efficiency in an unknown manner, we used well-defined saccharides for this purpose. As non-permeating CPAs, saccharides can preserve the cells during the thawing process by minimizing the osmotic shock (Wright DL, et al., Reprod. Biomed. Online 9, 179-186). It has been previously reported that raffinose in concentrations of 10-18% (w/v) is an effective CPA (Tada N, et al., J. Reprod. Fertil 89, 511-516). Other researchers have shown that melezitose and maltose offer acceptable cryoprotective effects (Hino T, et al., Reprod. Med. Biol 6, 229-233). Trehalose and sucrose may also reduce the intramembrane lateral stress, which leads to a practical cryopreservation approach (Yoon YH, et al., Biophys. J 74, 1949-1965). DMSO is a permeating CPA that inhibits the formation of intracellular spicular ice crystals. Our findings showed that cell viability is a function of the type and the concentration of CPAs. Furthermore, the freezing regime had a critical effect on cell viability. For many types of cells, there is a so-called “temperature danger zone,” which lies between −15° C. to −60° C. (Zhao G and Fu J, Biotechnol. Adv 35, 323-336). During the freezing/resuscitation processes, the cells pass this range of temperatures twice. Staying longer periods in this zone may exacerbate cell damages. In addition, slow freezing causes a gradual increase in the solute concentration of the intra- and extracellular solutions, leading to a change in osmotic pressure (Jang TH, et al., Integr. Med. Res 6, 12-18). On the other hand, the chance of intracellular ice crystallization and cell damage increases when the freezing procedure is too rapid (Baust JG, et al., Organogenesis. 5, 90-96). These opposite effects contribute to reducing cell viability, thereby narrowing the range of optimum cryopreservation.

As thoroughly reviewed before (John Morris G and Acton E, Cryobiology. 66, 85-92), the formation of sharp ice crystals at the ice nucleation point may jeopardize the viability of encapsulated cells. We found that the addition of CPAs decreased the sharpness and sizes of the ice crystals in the frozen hydrogel constructs. It was previously reported that the addition of CPAs lowers the freezing point of water and changes the ice crystals' microstructure (Jiang S, et al., Nat. Commun 10, 1-14). We observed a similar phenomenon for our GelMA-based cryoprotective bioinks. Avoiding the formation of spicular ice crystals is critical during freezing/thawing and plays a vital role in keeping the cells intact (Deller RC, et al., Nat. Commun 5, 1-7). In complement to the benefits of CPAs, the hydrogels' 3D network also reduces ice-crystal formation, thereby contributing to an intrinsic physical cryoprotective microenvironment for the cells (Kanmani P, et al., Biotechnol. Bioprocess Eng 16, 1106-1114). This fact is in great accordance with previous observations reporting that cell-laden hydrogel scaffolds containing CPAs offer a decent cryopreservative environment leading to high cell viability and cell functionality after revival (Cagol N, et al., Tissue Eng. Part C Methods 24, 20-31).

Intriguingly, we stored cryobioprinted cell-laden constructs for up to 3 months without any significant reduction in cell viability, proving the capability of our method to fabricate shelf-ready products that could sustain extended storage prior to usage. Apart from viability, the functionality of the cryobioprinted cells is also essential to determine whether this fabrication and storage process has imposed any permanent damage to the cells. We used two methods to demonstrate how different types of cells could remain functional after being cryobioprinted. First, the capability of hMSCs in differentiation was investigated. It was shown that similar to normal hMSCs, cryobioprinted hMSCs were able to successfully differentiate to osteoblasts, chondrocytes, or adipocytes when exposed to proper differentiation media. Furthermore, we demonstrated that cryobioprinted HUVECs and VEGF could maintain their ability to induce angiogenesis when investigated in the CAM assay. These in vitro cell differentiation studies and ex ovo CAM assays shed light on the efficacy of the cryobioprinting method as a functional approach for creating cell-laden implants or tissue models.

### Conclusion

In conclusion, cryobioprinting as a synergistic biofabrication method that combines the advantages of bioprinting and cryopreservation was developed. Our findings showed that cryobioprinting is a feasible method for fabricating storable cell-laden constructs to be used as the next generation of shelf-ready implants and tissue products. As the field of tissue engineering grows fast, these storable implants are envisioned to facilitate the clinical translation of the bioprinting method. Expanded applications towards laboratory and other usages, such as storable tissue models that can be readily shipped to desired sites, are also envisaged.

### Experimental Procedures

### GelMA Synthesis

Unless otherwise mentioned, all materials were purchased from Sigma-Aldrich. GelMA was prepared based on a previously developed protocol (Gong J, et al., Nat. Commun 11, 1-14). Briefly, porcine skin type-A gelatin was dissolved in PBS with a concentration of 10% (w/v) at 50° C. Methacrylic anhydride was then added drop-by-drop using a syringe pump until a concentration of 5% (v/v) was reached. The emulsion was mixed on a magnetic hot plate for 2 h at 50° C. to ensure homogeneity. GelMA was then diluted twice and dialyzed using distilled water at 40° C. for 5 days. The distilled water was changed every 12 h. The solution was filtered at 40° C. using a 0.22-μm Stericup-GP Sterile Vacuum Filtration System (Millipore). The filtered solution was then aliquoted into 25-mL lots and stored at —80° C. for 24 h. The frozen GelMA was lyophilized for 5 days at 0.2 mbar and 24° C. in a FreeZone Labconco TM freeze-dryer. The desired amount of lyophilized GelMA was then dissolved in a pre-mixed solution of PBS and 0.6% (v/v) 2-hydroxy-4-(2-hydroxyethoxy)-2-methylpropiophenone (Irgacure 2959, Sigma-Aldrich) as the photoinitiator. The resulting solution was used as the base bioink for cryobioprinting.

### Bioink Design

GelMA at a final concentration of 5% (w/v) was used as the main constituent of the bioink. DMSO, along with different saccharides listed in Table 1, were added as the CPAs. D-(+)-trehalose dihydrate, D-lactose, sucrose ultrapure, D-(+) raffinose pentahydrate, and D-(+)-melezitose hydrate were purchased from Alfa Aesar. The saccharides' concentration (0%, 4%, 8%, 12% (w/v)) and the DMSO concentration (0%, 5%, 10%, 15% (v/v)) were systematically studied to identify the optimal values based on cell viability results.

TABLE 1
The properties of the saccharides used in this study.

| | Molecular | Solubility at | Density | |
|---|---|---|---|---|
| Sugar | formula | 20° C. (g mL⁻¹) | (g cm⁻³) | Type |
| Trehalose | C₁₂H₂₂O₁₁ | 0.689 | 1.58 | Disaccharide |
| Lactose | C₁₂H₂₂O₁₁ | 0.216 | 1.52 | Disaccharide |
| Sucrose | C₁₂H₂₂O₁₁ | 2.100 | 1.59 | Disaccharide |
| Raffinose | C₁₈H₃₂O₁₆ | 0.203 | 1.80 | Trisaccharide |
| Melezitose | C₁₈H₃₂O₁₆ | 0.781 | 1.80 | Trisaccharide |
| Maltose | C₁₂H₂₂O₁₁ | 1.080 | 1.54 | Disaccharide |

### Cryobioprinting

A continuous extrusion bioprinter, Allevi 2 (3D Systems), was used for cryobioprinting. The Repetier-Host (Hot-World GmbH & Co. KG) and Cura (Ultimaker) software packages were employed to process the models and generate the corresponding G-codes. A self-designed and manually assembled freezing plate was used as the bioprinting substrate. The freezing plate temperature was set to −15° C. The needle size (27G), pressure (30 psi), and printhead speed (5 mm s⁻¹) were selected based on the printability results. The required time for printing each layer of an 8×8-mm² grid structure was approximately 8 s. To enhance the viscosity of the bioink, the cell-encapsulated GelMA/CPA was kept at 4° C. for 20 min before bioprinting. The samples were bioprinted on the freezing plate and stored in −80° C. or liquid nitrogen.

### Ice Crystal Imaging

The hydrogel groups were cast on the freezing plate to study the effect of DMSO concentration on ice crystal-formation. A volume of 25 μL of the precursor was deposited on the freezing plate. The process was recorded using a brightfield microscope eyepiece camera (GXCAM-D800—GT Vision Ltd.) mounted on a Leica EZ4 stereomicroscope (Leica Camera AG). Different concentrations of DMSO and melezitose were used in the hydrogels. The ice crystal-formation was quantified by processing the video frames using an open-source code (Jiro D., Grabit: Extract (pick out) data points off image files. MATLAB Central File Exchange) and a customized script in MATLAB (Mathworks).

### Splat Ice Recrystallization-Inhibition Assay

The splat cooling assay was performed following the previously developed protocols (Georgiou PG, et al., J. Am. Chem. Soc 143, 7449-7461). Briefly, 10 μL of samples with different DMSO concentrations was added between two glass coverslips. The coverslips were placed on the freezing plate (Tₚ=−20° C.) for 30 min. Images were then obtained using an optical microscope (Nikon) and processed by ImageJ (National Institutes of Health) to determine the ice crystal size in each group.

### Heat-Transfer Simulations

COMSOL Multiphysics® was employed to simulate heat-transfer in cryobioprinting a two-layer 8×8-mm² grid structure. The layers were generated by sweeping a 2-mm-thick rectangle over a defined bioprinting path. The simulation parameters are listed in record. Heat-transfer in the porous hydrogel was modeled by defining proper boundary conditions and using time-marching schemes. The layers were divided into 45 sections. The computational domain was discretized using an unstructured grid of free tetrahedral meshes with a size between 0.28 mm and 0.12 mm

The top and the side boundaries were defined so that the convection heat-transfer with the ambient medium was taken into account. The cryobioprinting procedure was simulated by imposing a step-function temperature decrease on the bottom surface of the bioprinted section, which contacts the freezing plate. The bottom surface of successive sections was exposed to the low temperature, i.e., the Tₚ, to simulate cryobioprinting deposition kinematics. By considering the printing speed, the sections were exposed to the low temperature every 0.16 s, one at a time. After the first layer was entirely deposited, the second layer was assembled section by section on top of the first layer. For the second layer, the continuity condition was applied to equate the temperature of the second layer's bottom surface with that of the first layer's top surface. As a simplifying assumption, the phase-change from liquid to solid was neglected to decrease the computation cost.

### Cell Culture

Seven types of cells, namely NIH/3T3 fibroblasts, C2C12 skeletal myoblasts, HepG2 hepatocellular carcinoma cells, MCF-7 breast cancer cells, SMCs, HUVECs, and hMSCs, were separately cultured. Dulbecco's modified Eagle's medium (DMEM, Thermo Fisher Scientific) enriched with 10% (v/v) FBS (Thermo Fisher Scientific) was used for culturing NIH/3T3, C2C12, HepG2, and MCF-7 cells. SMC growth medium, which was supplemented with 5% (v/v) FBS, 0.1% (v/v) insulin, 0.2% (v/v) human fibroblast growth factor-basic (hFGF-b), 0.1% (v/v) gentamicin sulfate-amphotericin (GA-1000), and 0.1% (v/v) human epidermal growth factor (hEGF) (Lonza Biologics), was used for culturing SMCs. HUVECs were cultured in endothelial cell growth medium supplemented with 2% (v/v) FBS, 0.04% (v/v) hydrocortisone, 0.4% (v/v) hFGF-b, 0.1% (v/v) VEGF, 0.1% insulin-like growth factor-1 (IGF-1), 0.1% (v/v) ascorbic acid, 0.1% (v/v) hEGF, 0.1% (v/v) GA-1000, and 0.1% (v/v) heparin (Lonza Biologics). Mesenchymal stem cell growth medium supplemented with 0.1% (v/v) GA-1000, 2% (v/v) L-glutamine, and 10% (v/v) mesenchymal cell growth supplement (Lonza Biologics) was used to culture hMSCs. The cells were cultured in T75 flasks at 37° C. in a 5% CO₂ humidified incubator. The media were changed every 3 days until confluency of approximately 80% was achieved. After removing the media and washing with Dulbecco's PBS (DPBS) 1×(Alfa Aesar), the cells were dissociated using 0.05% (v/v) trypsin-ethylenediaminetetraacetic acid (trypsin-EDTA) (Thermo Fisher Scientific).

### Cryopreservation

Ninety-six groups of GelMA samples (four DMSO concentrations, four saccharide concentrations, and six types of saccharides) were studied to identify the optimal CPA configuration. C2C12 cells were encapsulated in the GelMA/CPAs precursor. A volume of 50 μL of the cell-laden precursor was directly cast on a freezing plate in aseptic conditions. The frozen samples were immediately stored at −80° C. or in liquid nitrogen (−196° C.) for cryopreservation. On day 3, the samples were removed from cryogenic storage and directly crosslinked via UV exposure. The samples were washed in PBS to remove any excess CPAs in preparation for subsequent use.

### Cell Viability

Cell viability was quantified within unthawed cell-encapsulated samples to identify the optimal combination of CPAs. Following the crosslinking and washing steps, the hydrogel samples were cultured for a definite period and then stained using calcein-AM and ethidium homodimer-1 (Thermo Fisher Scientific) per the provider's protocol. An inverted Eclipse-Ti fluorescence microscope (Nikon) was employed to image the live and dead cells in green and red channels, respectively. The ratio of live cells to the total captured cells in random regions of the samples was determined through processing the fluorescent images using ImageJ.

### Cell Differentiation

The hMSCs were encapsulated in the selected group of GelMA/CPA to be used as the bioink. The cryobioprinted samples were stored at −80° C. overnight and were then transferred to liquid nitrogen for cryopreservation. On day 14, the samples were UV-crosslinked and revived. After 3 days of culture, the three types of differentiation media were separately added. The constructs were cultured in DMEM (4.5 g L⁻¹ of D-glucose) supplemented with 1-μM dexamethasone, 5-μg mL⁻¹ insulin, and 50-μM indomethacin (Ying G, et al., Adv. Funct. Mater 30, 2003740) to induce adipogenesis. Osteogenesis was conducted by culturing the constructs in DMEM (1 g L⁻¹ of D-glucose) supplemented with 10-mmol L⁻¹ β-glycerophosphate, 300-μmol L⁻¹ L-ascorbic acid, and 100-nmol L⁻¹ dexamethasone ((Ying G, et al., Adv. Funct. Mater 30, 2003740). For chondrogenic differentiation, the constructs were cultured in DMEM (4.5 g L⁻¹ of D-glucose) supplemented with 50-μg L⁻¹ ITS+Premix Tissue Culture Supplement, 100-nmol L⁻¹ dexamethasone, 1-μM ascorbate-2-phosphate, and 10-ng mL⁻¹ transforming growth factor-β1 (Solchaga LA, et al., Methods Mol Biol. 698, 253-278). At certain time points, i.e., day 7, dayl4, and day 21, Oil Red O, Alizarin Red S, and Alcian blue (all from ScienCell Research Laboratories) were used for staining the samples and characterizing adipogenesis, osteogenesis, or chondrogenesis.

### Immunostaining

For immunofluorescence staining, the anti-PPARγ, anti-RUNX2, anti-osteocalcin, or anti-SOX-9 antibodies (all from Abcam) were diluted in PBS to achieve a final concentration of 200 pg L⁻¹ and incubated overnight at 4° C. for adipogenesis, osteogenesis, or chondrogenesis. The samples were washed with PBS and then stained with the corresponding secondary antibodies. The samples were washed again with PBS, and fluorescence micrographs were captured using an inverted fluorescence microscope.

### CAM Assay

The ex ovo chick CAM culture was undertaken following the guidelines used in the previous studies (Mangir N, et al., ACS Biomater. Sci. Eng 5, 3190-3200). For preparing the VEGF group, the cryoprotective bioink was supplemented with 100 ng mL⁻¹ of VEGF (PeproTech) (Wu Y, et al., Ann Biomed Eng. 42, 1024-1036). The eggshells were carefully cracked to start the ex ovo culture 3 days after incubation. The cryobioprinted scaffolds were implanted in chick embryo CAM on day 7. The samples were then incubated for another 7 days until they were collected for imaging. We used 10% (v/v) formalin to fix the samples for inspecting the angiogenesis. An optical camera (EOS 60D, Canon) was used for photographing. The discernible BV lengths on the cryobioprinted constructs were measured using the NeuronJ tracing toolbox of ImageJ to quantify the extent of vascularization (Mangir N, et al., ACS Biomater. Sci. Eng 5, 3190-3200).

### Histology

The samples collected from the CAM assays were fixed in 10% (v/v) formalin for 24 h. The fixed samples were then dehydrated, embedded in paraffin wax, and then serially sectioned (5 μm in thickness). Goldner's trichrome staining involved the use of Weigert's hematoxylin, ponceau-fuchsin-azophloxin (erythrocytes), phosphomolybdic acid (cytoplasm), and light green (collagen) (Cidonio G, et al., Biofabrication. 11, 35027). The BV density and the ratio of BV-to-tissue areas in the different groups were calculated (Choi SW, et al., Adv. Healthc. Mater 2, 145-154).

### Statistical Analyses

All data are expressed as means ±standard deviations Minimum triplicates were used for all the experiments. One-way analysis of variance (ANOVA) was used to compare the effect of different CPAs on cell viability. The two-tailed paired or unpaired student's t-test was used for determining the significantly different values. A P-value of 0.05 was considered as the threshold.

### Example 2: Support Bath-Free Vertical Extrusion Cryo(Bio)Printing for Anisotropic Tissue Manufacturing

Three-dimensional (3D) bioprinting technology has attracted increasing attention in tissue fabrication over the past couple decades. As arguably the most common technique, conventional 3D extrusion bioprinting is mainly used for constructing volumetric structures in a layer-wise manner Although the layer-by-layer bioprinting method is functional in a majority of cases, there are limitations associated with creating anisotropic tissues, such as muscle fibers and nerve fibers that heavily rely on cellular alignment for their physiologies. Therefore, developing a versatile strategy that allows convenient 3D bioprinting synergized with simultaneous generation of structural anisotropy is essential for these applications.

Numerous studies have shown that porous hydrogel scaffolds can potentially enhance cell spreading and proliferation. Ying et al., Adv Funct Mater 2020, 30. In particular, ice-templating, one of the most widely utilized techniques for the fabrication of materials with anisotropic microchannels, allows control over pore morphologies by controlling directional ice-formation in a suspension of solute(s). Seifert et al., Advanced Engineering Materials 2021, 23, 2001417. During the freezing process, ice crystals form and propagate through a set direction within the biomaterial solution. When the construct crosslinks and thaws, the melted ice crystals form interconnected anisotropic microchannels within the scaffold. Importantly, previous studies have clearly demonstrated that the presence of anisotropic microchannels enhances alignment of post-seeded cells. Bai et al., Acta Biomater 2015, 20, 113. However, the freezing process, including directional freezing, of the biomaterials, will inevitably decrease viability of encapsulated cells. To this end, it is necessary to combine the biomaterials with cryoprotective agents (CPAs) to increase cell viability during and after the ice-templating process.

Dimethyl sulfoxide (DMSO) is one of the most widely used CPAs that can minimize damage on the cell membrane by hindering the intracellular and extracellular ice-crystallization under cryogenic conditions. Chang et al., Nat Biomed Eng 2021. Saccharides have also been widely used as naturally derived cryoprotectants. Rodrigues et al., Cryobiology 2008, 56, 144. Due to their high molecular weights, saccharides do not penetrate the cell membranes and would merely act on the extracellular surfaces of cells. Furthermore, saccharides can reduce the amount of water in contact with cells by increasing the osmolarity of the extracellular medium. Among various disaccharide, melezitose has shown promising cryoprotective effects (Hino et al., Reprod Med Biol 2007, 6, 229); therefore, it was chosen, either to act alone or in combination with DMSO, as the CPA.

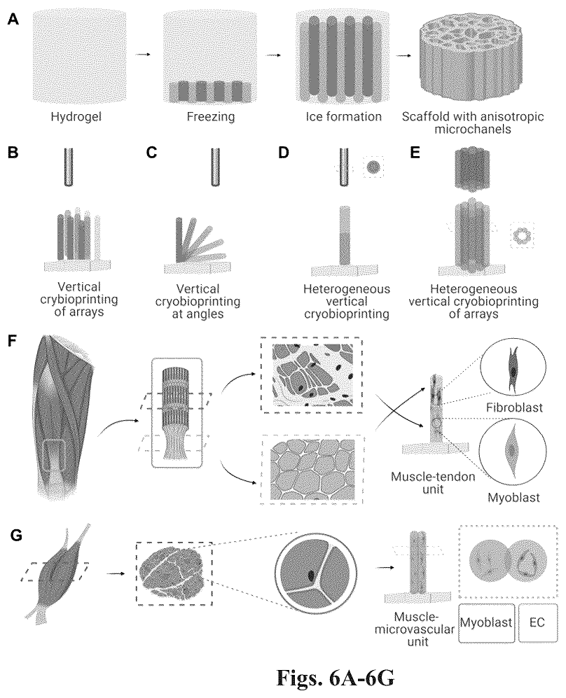

Here, we report an enabling technique that was largely unexplored before, termed vertical 3D extrusion cryobioprinting, that combines 3D extrusion bioprinting and ice-templating methods to fabricate vertical, high-aspect ratio, cell-laden hydrogel constructs with interconnected, anisotropic, gradient microchannels. As schematically briefed in FIG. 6A, a gelatin methacryloyl (GelMA)-based hydrogel scaffold containing anisotropic microchannels could be formed after ice-templating. The vertical 3D cryobioprinting technique was shown for its ability to produce pillar arrays (FIG. 6B), filaments at oblique angles (FIG. 6C), multi-material filaments (FIG. 6D), and scaffolds with arrays of heterogeneous filaments (FIG. 6E). As proof-of-concept demonstrations, we further utilized the vertical 3D cryobioprinting technique to fabricate the muscle-tendon unit (MTU) (FIG. 6F) with C2C12 myoblasts and NIH/3T3 fibroblasts, as well as the muscle-microvascular unit (MMVU) (FIG. 6G) with C2C12 myoblasts and human umbilical vein endothelial cells (HUVECs). Our results indicated that the bioink, consisting of GelMA and CPAs, could be effectively used in vertical 3D cryobioprinting to enable cell encapsulation at high viability levels. With the help of the interconnected, anisotropic, gradient microchannels formed by directional freezing during the vertical 3D cryobioprinting process, desired cellular alignments were also realized. These fabricated tissue constructs may find a plethora of applications in muscular tissue engineering and beyond.

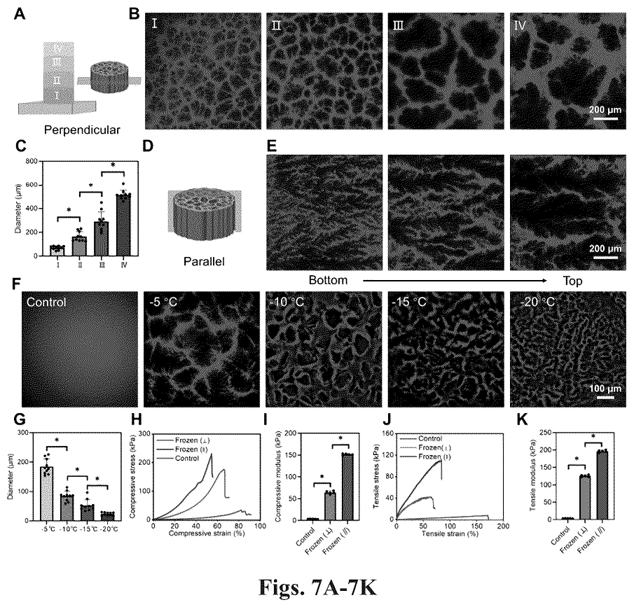

A thermoelectric cooler was used to precisely control the temperature on the substrate surface via adjusting the input voltage (FIG. 7A). Using polydimethylsiloxane (PDMS) molds, the 7.5% (w/v for all expressions unless otherwise indicated) rhodamine B-conjugated GelMA solution was directionally frozen using −20° C. as the substrate temperature. The samples were then exposed to UV light to fully crosslink. During the ice-templating process, lamellar ice crystals would start to grow from the substrate surface throughout the GelMA construct vertically. Shao et al., Adv Mater 2020, 32, e1907176. To confirm, the scaffolds were subsequently sliced into four pieces with a thickness of 1 mm each and imaged using a fluorescence microscope at the cross-sections. Honeycomb-like networks of microchannels at the cross-sections were apparent after the directional ice-templating process, showing gradient pore sizes across the height of the construct (FIG. 7B). In previous studies, it was also found that the temperature gradient can control the diameter of the microchannels, which is consistent to the current study. Stuckensen et al., Advanced Materials 2018, 30, 1706754. Following the measurements of the microchannel sizes directly from the fluorescence micrographs, it was quantified that the diameter of the microchannels increased from 70.68±15.64 μm to 513.63±39.88 μm along the ice-templating direction from bottom to top (FIGS. 7C). Under the pressure of the lamellar ice crystals during the directional freezing process, the GelMA chains self-coalesced and became separated from the initial homogeneous phase, which in turn formed the micrometer-scale aligned porous microchannels, as revealed also in the sagittal views (FIGS. 7D-E) Hua et al., Nature 2021, 590, 594.

Through the diffusion assay using a blue dextran solution, the interconnectivity of the microchannels of the ice-templated GelMA hydrogel was examined It was observed that the blue dextran solution could easily transport through the directionally frozen GelMA hydrogel disk but could not readily permeate the control GelMA scaffolds. The substrate temperature was further adjusted from −5° C. to −20° C. to study the effect of substrate temperature on the pore size of the resulting scaffolds (FIG. 7F). As the substrate temperature was decreased from −5° C. to −20° C., the pore size in the bottom layer decreased from 184.81±26.19 μm to 23.80±5.0 μm (FIG. 7G). The transparency of the GelMA scaffolds at different freezing temperatures was also compared. As the freezing temperature was reduced, the transparency of the scaffolds gradually decreased as well, due to the formation of denser porous structures inside. In addition, we investigated the influence of GelMA concentration on the diameter of the microchannels, wherein as the concentration of GelMA was increased, the microchannel size decreased.

Directional ice-templating results in increased local concentrations of GelMA surrounding the ice crystals, leading to the formation of anisotropic microchannels. The mechanical properties of the hydrogel constructs with anisotropic microchannels were investigated with compressive and tensile testing. The 7.5% GelMA hydrogels prepared via directional freezing showed unique anisotropic mechanical properties (FIG. 7H-K). It was found that when 7.5% GelMA was directionally frozen at −20° C., the compressive modulus parallel to the freezing direction of the scaffolds (151.56±1.56 kPa) increased significantly than that perpendicular to the freezing direction (64.32±3.47 kPa), though both much larger than that of the control samples (3.88±0.31 kPa) (FIG. 7H-I). Also, the tensile modulus parallel to the freezing direction of the directionally frozen GelMA scaffolds at −20° C. increased to 196.40±3.19 kPa, which was significantly higher than that perpendicular to the freezing direction (125.30±3.18 kPa) and was almost 55 times higher than that of the control samples (3.61±0.29 kPa) (FIG. 7J-K). Together, such an ice-templating method could increase the local concentrations of GelMA chains with simultaneously enhanced anisotropic microchannels and mechanical properties in the GelMA hydrogel constructs.

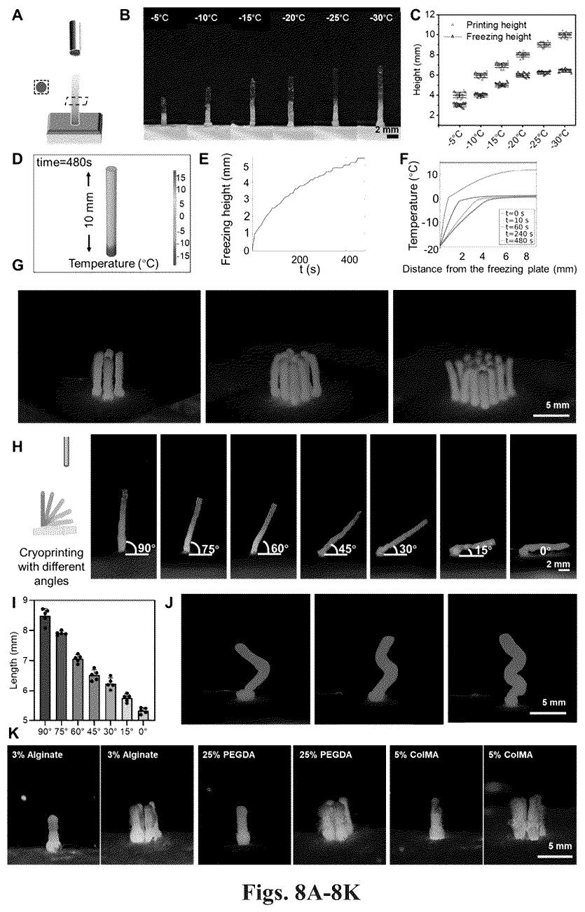

FIG. 8A schematically illustrates the vertical cryoprinting process of a GelMA pillar. The bioink is extruded onto the freezing plate through the nozzle to produce a vertically oriented structure. Although the printing of such vertical structures may also be possible when using an ambient freezing chamber (Leu et al., CIRP Annals 2012, 61, 223), anisotropic microchannels would not form as in our case with the use of the freezing plate at the bottom. In addition, an ambient freezing chamber may cause freezing of the bioink at the cartridge, potentially making the printing process difficult. In our setup, the temperature gradually decreases along the vertical direction from the bottom to the top. By adjusting the input voltage of the freezing plate, we were able to accurately control the freezing plate temperature from −5° C. to −30° C. (FIG. 8B), which accordingly, would affect the freezing speed and the printing speed. Of note, the bioink mixture is a eutectic system, and due to its colligative properties, it may have a freezing point of lower than 0° C. depending on the type and concentration of its components. The effect of gelatin concentration on the freezing point has been previously investigated in another study. Ueno et al., Bioscience, Biotechnology, and Biochemistry 2009, 73, 2478. Accordingly, for the case of 7.5% GelMA that we used primarily as the bioink, the freezing point was estimated at −0.45° C. As shown in FIG. 8B, when the freezing plate temperature was reduced, the heights of freezing and cryoprinting of GelMA constructs both increased. When the temperature of the freezing plate was −5° C., the average freezing height of the pillar was 2.99±0.17 mm As the temperature declined to −30° C., the average height of freezing increased to 6.43±0.15 mm (FIG. 8C). In addition, to explore how ambient temperature might be affect the cryoprinting process, we further made an enclosure to maintain the ambient temperature at 4° C. instead of the room temperature (RT) otherwise used. We found that there was no noticeable difference in the height of freezing with or without a chamber. Similarly, the heights of cryoprinting increased when lower temperatures were used for the freezing plate. We also assessed the cryoprinting performances of GelMA with different concentrations (2.5%, 5%, 7.5%, 10%, 12.5%, and 15%) on the freezing plate (−20° C.).

Since the bioink for vertical 3D cryoprinting freezes immediately after contacting with the freezing plate, those with very low viscoelasticity values, such as the 2.5% GelMA bioink, were conveniently cryoprinted vertically, which would be almost impossible in conventional extrusion bioprinting without freezing for a bioink containing this low polymer concentration. Different sizes of the nozzles (18G, 20G, 21G, 22G, 23G, and 25G) were also evaluated for vertical 3D cryoprinting. We successfully tuned the diameter of the cryoprinted GelMA pillar structures, where it was further observed that the nozzle diameter did not seem to affect their freezing heights although the printing heights suggested a decreasing trend when the nozzle size was reduced. It was possibly because as the nozzle size was decreased, the GelMA structure was more likely to break during the printing process towards the top and therefore would cause a decreasing height of the cryoprinted pillar. The swelling ratio of the cryoprinted structures was also measured, and the swelling ratio was approximately 12% at 1 day after printing.

COMSOL Multiphysics® was used to simulate phase-changing heat-transfer in the vertically cryoprinted structures. FIG. 8D shows the simulation of heat-transfer during the 480 s of vertical cryoprinting. As the bioink is in direct contact with the freezing plate (−20° C.), the heat transfers from the bioink and it gradually freezes upward. It is worth mentioning that the cooling rate in the bioink decreased as the construct height was increased; the higher the vertical structure, the longer it took for the sample to freeze (FIG. 8E). In FIG. 8F, the predicted temperatures over the height of the construct at different time points are plotted. The results portrayed that the height of freezing of the bioink construct nicely matched those of the simulation model, which is an indicator of synergy between simulation and experimental results. Specifically, we repeated the heat-transfer simulation for 5-mm pillars to directly compare the results with experimental data. When the 5-mm pillars were vertically cryoprinted, the freezing height reached 1.9 mm in 36 s, which was close to the value obtained from the simulation, i.e., 1.7 mm When 10-mm vertical pillars were cryoprinted at −20° C. (FIG. 8B), the ultimate freezing height reached roughly 5 mm, which was also very close to the simulation results in FIG. 8E, further providing good agreement between experimental data and simulation results regardless of the pillar height.

Vertical GelMA pillar arrays, featuring 2-by-2, 3-by-3, and 4-by-4 filaments, were successfully cryoprinted to further demonstrate the capability of the vertical 3D-cryoprinting technique (FIGS. 8G). In addition, oblique GelMA structures cryoprinted at a wide range of angles, from 90° to close to 0° varying every 15°, were obtained at −20° C. of the freezing plate (FIGS. 8H). We also measured the length of the cryoprinted structures and concluded that the printable length was dependent on the angle of the pillar (FIG. 8I). The printable length of the construct at 90° was 8.48±0.25 mm, consistent with our previous results, while it was 5.32±0.08 mm for 0°. The direction and diameter of the microchannels of the cryoprinted vertical and 45° filaments were also compared, where no significant differences were found between the samples in both of these parameters. Although nanocomposite hydrogels can be extrusion-printed in 3D freeform (Chen et al., International journal of bioprinting 2020, 6, 258) as well as using vat polymerization-based method (Ng et al., Biofabrication 2020, 12, 022001), our vertical 3D-cryoprinting technique was more convenient in producing various vertical GelMA structures (FIGS. 8J) and that were spiral, without necessitating bioink or instrument complexity. In addition to cryoprinting GelMA vertically, bioinks without thermogelling properties (3% alginate, 25% poly(ethylene glycol)-diacrylate (PEGDA), and 5% collagen methacryloyl (ColMA)) were also successfully cryoprinted in the vertical direction as single pillars and as 2-by-2 pillar arrays (FIGS. 8K).

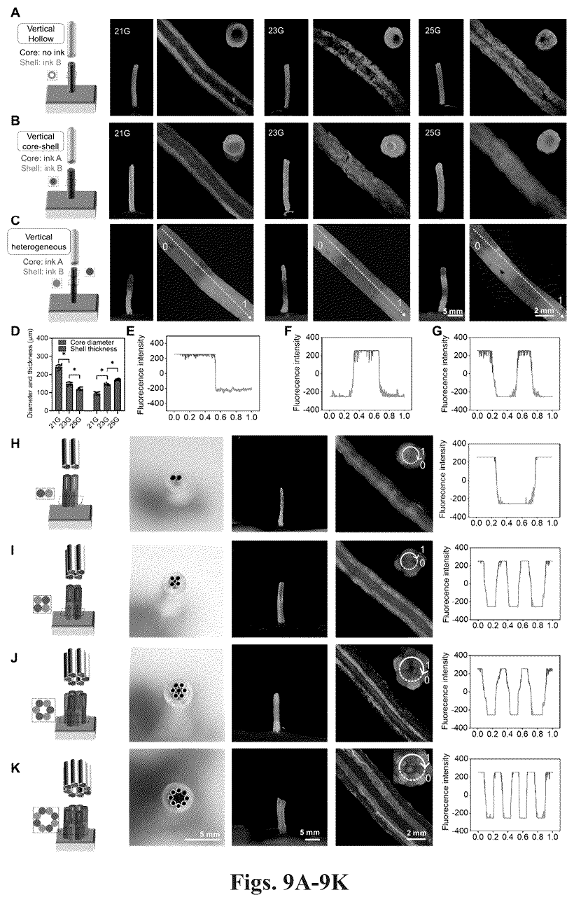

Another unique feature of vertical 3D cryoprinting is the ability to create sophisticated vertical structures such as those hollow or/and multi-material in nature. We first utilized coaxial nozzles to extrude the GelMA bioink containing green fluorescent microbeads in the sheath layer, and cryoprinted hollow vertical structures of different core diameters with the help of 21, 23, and 25G inner needles, where the outer layer of the nozzles was fixed at 18G (FIG. 9A). The hollow core diameters of the resulting constructs were measured using the fluorescence microscopic images. As shown in FIG. 9D, the core diameters were 241.94±12.88 μm, 148.69±8.63 μm, and 119.99±8.08 μm, respectively, for 21, 23, and 25G nozzles.

In addition, core-shell vertical constructs made of multiple materials could be cryoprinted when two different bioinks were loaded in the core and shell during the fabrication process (FIG. 9B). Although creating fibers with multi-layer structures has been previously investigated (Q et al., Adv Mater 2018, 30, e1706913), our unique vertical cryoprinting method introduces additional flexibility to their production with enhanced structural controls. The shell thicknesses of the cryoprinted vertical core-shell structures were measured at 95.18±9.33 μm, 145.36±5.20 μm, and 171.76±5.24 μm, respectively, for 21, 23, and 25G nozzles (FIG. 9D). More interestingly, a programmable distribution of the multiple materials along the vertical direction of the solid filaments could be achieved by periodically switching the core and shell channels ON/OFF (FIGS. 9C). The corresponding fluorescence intensity profiles indicated that the various components within a given cryoprinted vertical structure were physically well-separated (FIG. 9E-G).

Beyond vertical cryoprinting of hollow, core-shell, and heterogeneous structures, we also designed a group of nozzles containing 1, 2, 4, 6, and 8 bundled channels for parallel vertical 3D cryoprinting (FIGS. 9H-K). The red and green colors represent different bioinks extruded from the different channels. Through this enabling nozzle system, vertical structures containing up to 8 bioinks were able to be fabricated. Similarly, the fluorescence intensity profiles demonstrated the well-defined multi-material compositions in the corresponding vertically cryoprinted constructs. Nevertheless, because the structure undergoes gradient cooling, the part near the cold plate freezes first and the part at the top freezes later, and therefore the connections in the bundles might be different among different sections of the bundle.

One exceptional feature in directional ice-templating within hydrogel scaffolds is the formation of anisotropic microchannels possibly with gradient sizes, which facilitate proper cell growth and designed alignment. Liang et al., Adv Mater 2021, e2102011. As previously stated, in vertical 3D cryoprinting, the cooling rate changes in relation to the height of the sample deposited; thus, dissimilar ice crystals are formed, leading to the formation of microchannels with graded alignments and sizes. On the other hand, ice crystals can also damage cell membranes resulting in poor cell viability. Chaytor et al., Glycobiology 2012, 22, 123. Maintaining cell viability during the ice-templating process is, therefore, a key factor in designing bioinks for cell-laden vertical cryoprinting, i.e., vertical cryobioprinting.

The C2C12 myoblast-laden cryobioprinted samples containing DMSO and melezitose were subjected to Live/Dead assays on Days 1, 3, and 7. The 7.5% GelMA bioinks containing different concentrations (5%, 10%, and 15%) of DMSO were initially used to gain understanding on its cryoprotectant effect. The optimum concentration of DMSO was found at 10% for maximizing cell viability in the absence of the saccharide. GelMA bioinks containing different concentrations (4%, 8%, and 12%) of melezitose in the absence of DMSO were separately used for optimizing the melezitose effect. The results suggested that 8% melezitose could lead to the highest cell viability, and further increasing the melezitose concentration had no significant benefit. The CPA-free control group generally suggested the lowest cell viability values across the 7 days of culture, while the samples containing a mix of DMSO and melezitose showed the highest cell viability. Therefore, the GelMA bioink combined with 8% melezitose and 10% DMSO was determined as the optimum formulation for cell-laden vertical cryobioprinting, and used in the following cellular experiments. Of note, the viability of the C2C12 cells at Day 1 was approximately 60% in the presence of both the optimal DMSO (10%) and the optimal melezitose (8%) conditions. However, under similar conditions, the viability of the C2C12 cells at day 1 was observed at 80%. Such an inconsistency might be caused by the different passage numbers of the different batches of cells or other inevitable differences in the experiment processes. While we felt that these inconsistencies were within acceptable error ranges, we always had control groups in these experiments to maximize the validity of the results.

Furthermore, the more fragile human mesenchymal stem cells (hMSCs) were also evaluated to verify the cryobioprinting method and cryopreservation efficacy using the optimum bioink formulation. Further data suggests that the cell viability was approximately 79.2%, 84.5%, and 83.9% after 1 day, 7 days, and 14 days of culture following cryobioprinting, respectively. In a previous study, researchers bioprinted 3% alginate with osteoblast-like cells at −40° C. to −2° C. Ahn et al., Journal of Materials Chemistry B 2014, 2, 2773. The results showed that cell viability was less than 40% when the temperature was lower than −20° C. Differently, in the current study, we illustrated that directional freezing in combination with cryobioprinting could generate anisotropic microchannels and induce desired cell alignment, as we will show below, which has not been reported before.

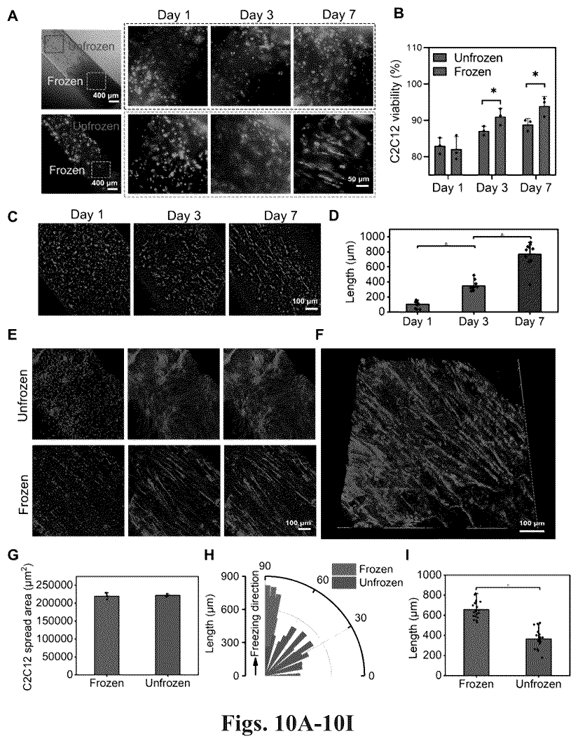

We studied the cell viability in the frozen and unfrozen areas of the same vertically cryoprinted filament surrounding the junction point (FIG. 10A). The unfrozen section was incorporated in this scenario with the aid through intentionally cryobioprinting a longer filament beyond the frozen length. It was clear that C2C12 cell viabilities in both frozen and unfrozen segments were 82% on Day 1 (FIG. 10B), proving that the cryoprotective bioink could effectively reduce the adverse effects of ice crystals on cell viability during the cryoprinting process. In addition, cell viability in the frozen segment was found to be higher than that in the unfrozen areas on Days 3 and 7 (FIG. 10B); on the 7th day, the cell viability in the frozen area was 94.01±2.34%, which was 5% higher than that in the unfrozen area (88.90±1.63%) (FIG. 10B), although both were deemed decently high and sufficient for our cryobioprinting applications. The explanation for the lower viability in the unfrozen part might be the extended presence duration of DMSO at above-freezing temperature, or possibly the presence of microchannels in the frozen portion that served to enhance the cell growth after vertical cryobioprinting during the subsequent culture period.

The myotube spreading in the vertically 3D-cryobioprinted hydrogel scaffolds on Days 1, 3, and 7 was examined to demonstrate the advantages of the gradient microchannel configuration (FIGS. 10C), the timeline of which is widely used in literature for assessing engineered muscular tissues. We found that C2C12 cells could differentiate into well-aligned myotubes within these microchannels. The myotubes' length started from 100.39±48.79 μm (per microscopic view) on Day 1 and reached 636.36±68.19 μm on Day 7 (FIG. 10D). Similarly, the cell spreading area gradually increased from 50,579.00±1,612.88 μm2 (per microscopic view) to 80,227.66±1,460.71 μm². The spreading of myotubes in the frozen and unfrozen areas of a single vertically cryobioprinted filament was compared (FIG. 10E). The myotube alignment in the anisotropic microchannels in the frozen segment was further characterized via 3D reconstruction of the Z-stack images captured with confocal fluorescence microscope (FIG. 10F). It was indicated that there was no significant difference in the spread areas of the myotubes in the two segments on Day 7 of culture (FIG. 10G). Compared with the random distribution of the myotubes in the unfrozen area, most of the myotubes in the frozen area were oriented perpendicular to the cross-section of the filament (FIG. 10H), again confirming that the anisotropic microchannels within the frozen segment were able to support the alignment and fusion myoblasts. The anisotropic microchannels were also influential in the increase of the myotube length. Compared with myotube length in the unfrozen area, i.e., 363.19±88.53 μm, their length in the frozen area significantly increased to 655.48±83.04 μm (FIG. 10I). It should be noted that, other strategies, such as including fibers into bioinks to induce cell alignment and functions, have also been demonstrated before. Pendergast et al., Biofabrication 2021, 13, 044108. In comparison, our vertical cryobioprinting strategy could not only form the aligned microchannels to enhance cell alignment at the microscale but also allowed generation of macroscale aligned filaments. Moreover, the mechanical properties of the filaments would be significantly increased as well, in addition to the ability to cryogenically store these cryobioprinted vertical structures prior to their thawing and culture as needed under certain usage scenarios.

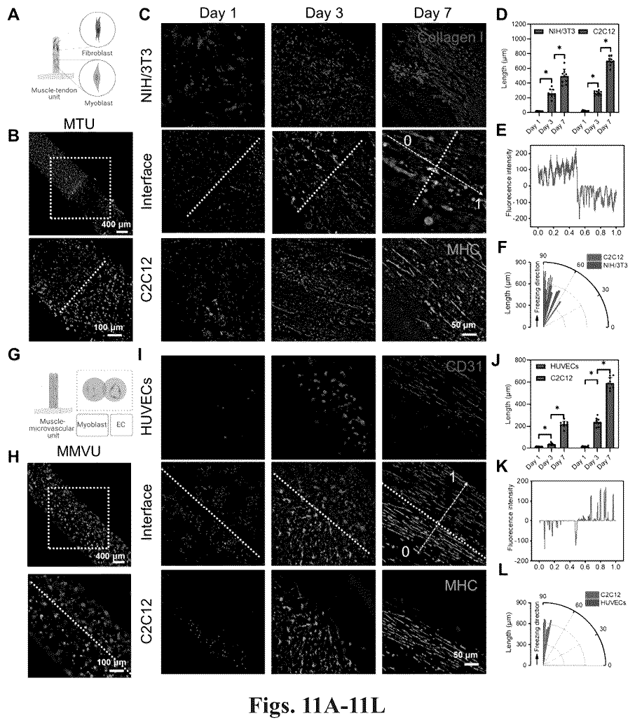

In muscular tissue engineering, one of the most challenging problems is the reconstruction of the muscle-tendon unit (MTU). There have been previous studies on bioprinted muscle (Hwangbo et al., Bioactive Materials 2022, 8, 57) or tendon (Toprakhisar et al., Macromolecular bioscience 2018, 18, e1800024) separately, but very few on employing cell-laden bioinks to create the MTU constructs through bioprinting, which still lacked localized topographical cues to allow necessary alignments. The key part of MTU is the muscle-tendon junction, which transmits the contraction force of the muscle through the tendon to the skeletal system. We took advantage of our vertical 3D cryobioprinting technique to propose a unique strategy for fabricating the muscle-tendon junction with preliminary biological characterizations. In this strategy, the junction was coincided with the frozen-to-unfrozen transition point of the vertically cryobioprinted filament, such that the myoblasts in the lower segment were highly aligned while the fibroblasts less aligned with a junction in between, mimicking the natural structure (FIG. 11A). Red and green fluorescence tracers were used to label NIN/3T3 fibroblasts and C2C12 myoblasts, respectively, to visualize the cell distributions in the vertically cryobioprinted MTU-like constructs (FIG. 11B). Fluorescence images in FIG. 11B clearly showed the junction between the muscle and tendon portions.

In addition, we used immunofluorescence to demonstrate the possible formation of the muscle-tendon junction, by labeling C2C12 and NIH/3T3 cells using anti-myosin heavy chain (MHC) and anti-collagen I antibodies, respectively (FIG. 11C). The increase in the area of MHC staining on the muscle side from 1,866.33±626.06 μm2 to 24,278.33±1,927.53 μm² after 7 days of culture, confirmed the formation of aligned myotubes. Similarly, on the tendon side, the increase in the area of collagen I from 1,983.33±368.46 μm² to 26,266.00±1,221.51 μm² was evident, suggesting that the fibroblasts were functional to secrete the main protein component of the tendon extracellular matrix (ECM). Moreover, the lengths of myotubes and fibroblasts gradually increased to 700.87±56.34 μm and 494.61±95.53 μm, respectively, during the 7 days of culture (FIG. 11D). Most importantly, the fluorescence intensity profiles indicated that the C2C12 cells and the NIH/3T3 cells were tightly connected within the microchannels at the interface region, which indicated that the vertical 3D cryobioprinting method was effective in the reconstruction of a preliminary muscle-tendon junction model (FIG. 11E). The orientation of the C2C12 cells and the NIH/3T3 cells were also assessed. The C2C12 cells in the lower area of the filament were oriented perpendicular to the cross-section of the filament, and the NIH/3T3 cells were less aligned in the upper region of the filament, which is close to the natural structure of the MTU (FIG. 11F).

On the other hand, the skeletal muscle is known to be highly vascularized. Microvasculature in the skeletal muscle is essential for supplying nutrients and oxygen as well as metabolic waste-removal. Tissue survival depends on oxygen supply, which is limited to a diffusion distance of up to approximately 200-300 μm from the supplying blood vessel. Researchers have embedded endothelial cells and myoblasts into hydrogel scaffolds to form vascularized grafts. Koffler et al., Proc Natl Acad Sci U S A 2011, 108, 14789. However, bioprinting of microvascularized skeletal muscle tissues still remains challenging in general. Accordingly, the interconnected, aligned microchannels in the vertically cryobioprinted hydrogel scaffolds were also exploited to fabricate the MMVU (FIG. 11G). We used red and green fluorescence tracers to respectively label HUVECs and C2C12 cells, and conducted vertical 3D cryobioprinting using a parallel, two-channel nozzle configuration (FIG. 11H). Fluorescence images showed a distinct interface between the muscular and vascular filaments (FIG. 11H).

Anti-MHC and anti-CD31 antibodies were then used to immunologically stain C2C12 cells and HUVECs, respectively (FIG. 11I). The HUVECs formed a dense microvascular network within the microchannels in the filament they were embedded in during vertical cryobioprinting, over the 7 days of culture, which were well-aligned with the adjacent myofibers in the parallel filament. It was also quantified that the average length of the microvessels reached 217.54±28.14 μm at Day 7 of culture, and their areas increased from 1,538.00±409.79 μm² to 14,748.33±792.04 μm² from Day 1 to Day 7. The myotube length increased from 15.01±5.56 μm on Day 1 to 591.36±48.48 μm on Day 7 (FIG. 11J), roughly matching those observed in the MTU experimentation. Similarly, the spreading area of the myotubes gradually increased following vertical cryobioprinting and subsequent culture. Finally, the fluorescence intensity profiles indicated that while the C2C12 cells and the HUVECs were separated in their respective filaments, they were closely arranged side-by-side showing signs of interactions and formation of the preliminary MMVU (FIG. 11K). The orientation assessment of the C2C12 myoblasts and HUVECs in the filaments further validated that both of them were aligned perpendicular to the cross-sections of the filaments (FIG. 11L).

In summary, we developed an enabling vertical 3D cryobioprinting technique by combining extrusion bioprinting, directional freezing, and cryopreservation. Compared with traditional pure GelMA hydrogel scaffolds, vertically 3D-cryoprinted constructs allowed generation of controllable, anisotropic, interconnected microchannels, which presented enhanced mechanical properties. The flexibility of our technique was demonstrated by vertical cryoprinting of sophisticated structures, core-shell structures, and multi-material structures. Contrary to conventional GelMA constructs, these vertically cryobioprinted could increase cell activities and regulate cell behaviors, showing advantages in certain applications. For example, highly aligned myotubes were induced to form for the reconstruction of muscular tissues by embedding C2C12 myoblasts during the vertical cryobioprinting process. We could also successfully construct the MTU or the MMVU by co-cryobioprinting myoblasts with fibroblasts or endothelial cells consecutively or in parallel. It is hence rationally anticipated that our vertical 3D cryobioprinting strategy may find broad usage in engineering a variety of tissues that feature oriented internal cellular and ECM arrangements.

### Materials and Methods

### GelMA Synthesis

Unless otherwise mentioned, all materials were purchased from Sigma-Aldrich. GelMA was synthesized as previously described. Ying et al., Adv Funct Mater 2020, 30. Briefly, 10 g of gelatin from porcine skin (Type A, —300 g Bloom, average Mw=90,000 Da) was dissolved in 100 mL of Dulbecco's phosphate-buffered saline (DPBS) by stirring at 50° C. at a concentration of 10%. 5-mL methacrylic anhydride (MA) was then added to the gelatin solution, in a dropwise manner, using a syringe pump until a concentration of 5% (v/v) was reached. The mixture was stirred on a magnetic hot plate for 2 h at 50° C. to ensure homogeneity. GelMA was then diluted twice and dialyzed using distilled water at 50° C. for 7 days. The distilled water was changed every 12 h. The solution was filtered at 40° C. using a 0.22-μm Stericup-GP sterile vacuum filtration system (Millipore). The frozen GelMA samples were then obtained by lyophilizing the solution and were stored at room temperature. When needed for use, a GelMA prepolymer solution was prepared by dissolving the freeze-dried GelMA at the desired concentration and the photoinitiator (LAP, Allevi) (0.5%) in DPBS at 50° C. The degree of methacryloyl-modification of GelMA is generally determined by 1H nuclear magnetic resonance, which is defined as the number of methacryloyl groups per available lysine found in the gelatin. The degree of methacryloyl-modification of GelMA used in our study was estimated to be approximately 50% according to a previous study of us. Yue et al., Biomaterials 2017, 139, 163. The conjugation of rhodamine B onto GelMA was based on a standard protocol. Xue et al., ACS Applied Bio Materials 2020, 3, 6908. In brief, 100 mg of rhodamine B were used to react with 1,000 mg of GelMA to obtain rhodamine B-conjugated GelMA.

### Preparation of Alginate, PEGDA, and ColMA Inks

Sodium alginate (Mw=33 kDa; low-viscosity, Sigma-Aldrich) was dissolved in PBS to a concentration of 3% (w/v). PEGDA (Mw=575 Da, Sigma-Aldrich) was dissolved in PBS to a concentration of 25% (w/v). Methacrylated collagen (ColMA, Advanced BioMatrix) was dissolved in PBS to a concentration of 5% (w/v).

### Bioprinting

A laboratory-designed mechanical (motor-driven) extrusion bioprinter was used for 3D bioprinting. A customized freezing plate was assembled to act as the collecting substrate. The temperature of the freezing plate was controlled via adjusting the input voltage. Depending on the freezing plate temperature, the printhead linear speed was tuned from 0.5 mm min⁻¹ to 2 mm min⁻¹. The extrusion volume was adjusted according to the printhead linear speed and nozzle diameter. After bioprinting, all samples were crosslinked through UV exposure.

### Coaxial Nozzles

Three coaxial nozzles were designed based on our previous reports. Pi et al., Adv Mater 2018, 30, e1706913. 18G needle was used as the shell, while the core needle was 21, 23, or 25G.

### Arrayed Multi-Channel Nozzles

Based on the number of extruding channels needed and inspired by our previous studies (Tang et al., Chemical Engineering Journal 2021, 407, 127187), the arrayed multi-channel nozzle bundles were designed to contain two-, four-, six-, and eight channels; all nozzle configurations utilized 21G needles. A central holder was added in the six- and eight-channel configurations to optimally align the needles in a coaxial manner and to keep the nozzle system stable.

### Heat-Transfer Simulations

COMSOL Multiphysics® was used to simulate phase-changing heat-transfer in the vertically 3D-bioprinted structures. A cylinder with the radius of 1 mm and the height of 2 cm was considered as the geometry of interest. The bottom surface of the cylinder was subjected to −20° C., i.e., the freezing plate temperature. For the sake of convergence, a smooth step-function temperature decrease was considered to apply this boundary condition. All the other surfaces were modeled using free-convection boundary condition. When the temperature at any location declines to a certain value, i.e., phase-change temperature, the material starts to change from liquid to solid. The geometry was discretized using physics-controlled, free tetrahedral meshes with a minimum element size of 0.08 mm A time-dependent study was conducted using a timestep of 9.6 s over 480 s, which is the required time to reach a stable temperature regime throughout the cylinder.

### Cell Culture

Three types of cells, namely C2C12 myoblasts, NIH/3T3 fibroblasts, and HUVECs were separately cultured. Dulbecco's modified Eagle's medium (DMEM, Thermo Fisher Scientific) enriched with 10% (v/v) fetal bovine serum (FBS, Thermo Fisher Scientific) was used for culturing NIH/3T3 and C2C12 cells. HUVECs were cultured in endothelial basal medium (EBM-2, Lonza Biologics) supplemented with the endothelial growth BulletKit and 1% (v/v) penicillin/streptomycin (P/S, Thermo Fisher Scientific). The cells were cultured in a 5% CO₂, humidified incubator at 37° C. The media were changed every other day, and the cells were passaged with 0.05% trypsin-ethylenediaminetetraacetic acid (trypsin-EDTA, Thermo Fisher Scientific) when a confluency of approximately 80% was achieved.

### Bioink Design and Preparation

7.5% (w/v) GelMA was used as the main constituent of the bioink. Different concentrations (5%, 10%, and 15% (v/v)) of DMSO, along with different concentrations (4%, 8%, and 12%) of D-(+)-melezitose hydrate were added as the CPAs. The cell density in the bioink used for vertical 3D bioprinting was 1×10⁶ mL⁻¹. For evaluating the maturation of the myoblasts in the scaffolds, the culture medium was replaced with the differentiation medium 2 days after bioprinting. The differentiation medium, containing DMEM with 2% (v/v) horse serum and 1% (v/v) P/S, was changed 3 times a week.

Cell Viability

Cell viability was assessed by staining with calcein-AM at 0.5 μL mL⁻¹ and ethidium homodimer-1 at 2 μL mL⁻¹ (Thermo Fisher Scientific). Cell viability imaging was done using an inverted Eclipse-Ti fluorescence microscope (Nikon). Cell viability was quantified by counting live and dead cells using the ImageJ software (National Institutes of Health). After loading the image, we would adjust the threshold to highlight all of the cells that need to be counted, and convert the image to a binary image. The number of live cells was then determined in the image. When counting the cells, the size of the elongated cells would affect the counting. The diameters of the elongated cells were set to more than 5 μm according to previous reports. McMahon et al., Am J Physiol 1994, 266, C1795. The Live/Dead image was subsequently merged. The number of cells could also be displayed. Triplicates were used for each study group.

### Immunofluorescence

After 7 days of culture, the bioprinted constructs were fixed in 10% (v/v) paraformaldehyde for 10 min The samples were then stained for expressions of specific markers using a standard immunofluorescence protocol. AlexaFluor® 594-labeled F-actin staining was used for characterization of cell morphologies in the cryobioprinted constructs. Rabbit anti-MHC primary antibody (1:200 dilution, Abcam) was utilized with an anti-rabbit, AlexaFluor® 488-labeled secondary antibody (1:400 dilution, Life Technologies) to identify muscle development. Mouse anti-collagen I primary antibody (1:200 dilution, Abcam) was utilized with an anti-mouse, AlexaFluor® 594-labeled secondary antibody (1:400 dilution, Life Technologies) to assess tendon development. Mouse anti-CD 31 primary antibody (1:200 dilution, Abcam) was utilized with an anti-mouse, AlexaFluor® 594-labeled secondary antibody (1:400 dilution, Life Technologies) to examine microvascular development. Nuclei of the cells were then counter-stained using 4′,6-diamidino-2-phenylindole (DAPI) solution (100 nmol L⁻¹) for 15 min, and the samples were washed three times. The constructs were observed and imaged with the fluorescence microscope (Nikon). A confocal fluorescence microscope (LSM880, Zeiss) was also used when necessary.

### Statistical Analyses

Statistical analysis was performed by IBM SPSS® Statistics software. All data are presented as the means±standard deviations. The unpaired t-test was used for two groups comparison, and one-way analysis of variance (ANOVA) with post hoc test was used for comparing three or more groups. P<0.05 was considered statistically significant.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of making a 3D-printed biomaterial, comprising
depositing a hydrogel comprising cells and a cryoprotectant from a 3D printer onto a freezing plate to form a frozen hydrogel filament;
depositing additional hydrogel comprising cells and a cryoprotectant from a 3D printer in contact with a previously placed frozen hydrogel filament to form a 3D-printed biomaterial comprising a plurality of frozen hydrogel filaments; and
removing the 3D-printed biomaterial from the freezing plate.

2. The method of claim 1, wherein the 3D-printed biomaterial is crosslinked.

3. The method of claim 2, wherein the 3D-printed biomaterial is crosslinked after being stored.

4. The method of claim 1, wherein depositing the hydrogel comprises extruding the hydrogel.

5. The method of claim 1, wherein the 3D-printed biomaterial is stored at 0° C. or less.

6. The method of claim 1, wherein the hydrogel is chilled to a temperature ranging from about 0° C. to about 10° C. before being deposited by the 3D printer.

7. The method of claim 1, wherein the hydrogel comprises gelatin methacryloyl (GelMA).

8. The method of claim 1, wherein the freezing plate has a temperature ranging from about −5° C. to about −30° C.

9. The method of claim 1, wherein the cryoprotectant is a saccharide cryoprotectant.

10. The method of claim 9, wherein the cryoprotectant is D-(+)-melezitose.

11. The method of claim 1, wherein the cells are myoblasts, fibroblasts, endothelial cells, or stem cells.

12. The method of claim 1, wherein the frozen hydrogel filaments are formed in the vertical direction.

13. The method of claim 1, wherein a plurality of frozen 3D-printed biomaterials are prepared and combined.

14. A 3D-printed biomaterial, made according to the method of claim 1.

15. The 3D-printed biomaterial of claim 14, wherein the 3D-printed biomaterial comprises microchannels.

16. The 3D-printed biomaterial of claim 14, wherein the 3D-printed biomaterial comprises a tissue scaffold or organoid.

17. The 3D-printed biomaterial of claim 14, wherein the 3D-printed biomaterial is a muscle tissue scaffold.

18. The 3D-printed biomaterial of claim 14, wherein the 3D-printed biomaterial is neuronal tissue.

19. The 3D-printed biomaterial of claim 14, wherein the 3D-biomaterial is crosslinked.

20. The 3D-printed biomaterial of claim 14, wherein the hydrogel is GelMA.

21. The 3D-printed biomaterial of claim 14, wherein the 3D-printed biomaterial comprises a plurality of different hydrogels.

22. The 3D-printed biomaterial of claim 14, wherein the cells are myoblasts, fibroblasts, endothelial cells, or stem cells.

23. The 3D-printed biomaterial of claim 14, wherein the 3D-printed biomaterial is frozen.
